(19) United States
(12) Patent Application Publication
(10) Pub. No.: US 2025/0223295 A1
(43) Pub. Date: Jul. 10, 2025

(54) SOLID STATE FORMS OF RUCAPARIB TOSYLATE

(71) Applicant: ASSIA CHEMICAL INDUSTRIES LTD., Tel Aviv (IL)

(72) Inventors: Dijana Skalec Samec, Jastrebarsko (HR); Valentina Travancic, Zagreb (HR); Damir Sahnic, Zagreb (HR)

(21) Appl. No.: 18/727,806

(22) PCT Filed: Jan. 11, 2023
(86) PCT No.: PCT/US2023/010592

§ 371 Date: Jul. 10, 2024

Related U.S. Application Data
(60) Provisional application No. 63/323,229, filed on Mar. 24, 2022.
(60) Provisional application No. 63/313,308, filed on Feb. 24, 2022.
(60) Provisional application No. 63/299,080, filed on Jan. 13, 2022.
(60) Provisional application No. 63/298,305, filed on Jan. 11, 2022.

(51) Int. Cl. C07D 487/06; A61K 31/55

(52) U.S. Cl. CPC C07D 487/06; A61K 31/55

(57) ABSTRACT

The present disclosure encompasses solid state forms of Rucaparib Tosylate, and pharmaceutical compositions thereof.

### FIELD OF THE DISCLOSURE

The present disclosure encompasses solid state forms of Rucaparib Tosylate, and pharmaceutical compositions thereof.

### BACKGROUND OF THE DISCLOSURE

Rucaparib chemical name is 8-fluoro-2-{4-[(methylamino)methyl]phenyl}-1,3,4,5-tetrahydro-6H-azepino [5,4,3-cd]indol-6-one, having the following chemical structure:

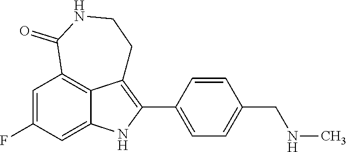 [SMILES: CNCc1ccc(-c2[nH]c3cc(F)cc4c3c2CCNC4=O)cc1]

Rucaparib is an inhibitor of the mammalian poly-adenosine 5′-diphospho-ribose polymerase (PARP) enzyme, indicated as monotherapy for the treatment of patients with deleterious BRCA mutation (germline and/or somatic) associated advanced ovarian cancer who have been treated with two or more chemotherapies. It was approved by the FDA as RUBRACA (Rucaparib Camsylate). Rucaparib/Rucaparib Camsylate is also being investigated as potential monotherapy or combination therapy for other cancers including prostate cancer, fallopian tube cancer and peritoneal cancer.

The compound is described in U.S. Pat. No. 6,495,541. A process for its preparation is described in U.S. Pat. No. 7,323,562, as well as in Org. Process Res. Dev., 2012, 16 (12), pp 1897-1904. U.S. Patent Publication No. 2004/0248879 also describes the following salts of Rucaparib: HCl, Mesylate, Phosphate, Glucuronate, Tartrate, Gluconate and Acetate. U.S. Pat. No. 7,268,126 describes crystalline forms and an amorphous form of Rucaparib Phosphate. U.S. Pat. No. 8,754,072 describes crystalline Camsylate and Maleate salts of Rucaparib. U.S. Pat. No. 9,045,487 describes Camsylate and Maleate salts of Rucaparib.

U.S. Patent Publication No. 2019/0389871 describes solid state forms of Rucaparib and Rucaparib salts.

International Publication No. WO 2018/140377 describes several solid-state forms of Rucaparib and Rucaparib salts. International Publication No. WO 2022/0015557 describes several solid-state forms of Rucaparib tosylate.

Rucaparib is administrated in a high-load dose, of 600 mg twice daily; available tablets strength are 200 mg, 250 mg, and 300 mg.

International Publication No. WO 2016/028689 describes a tablet of Rucaparib Camsylate in certain active pharmaceutical ingredient (“API”) amounts. According to this application, at high drug loading, the contribution of the physical properties of the API to the manufacturability of a formulation is predominant. According to the applicant, high drug loading requires compressibility properties which are contributed due to the API. In that application, it is stated that Rucaparib Camsylate has advantageous properties with respect to compressibility and that it is possible to manufacture tablets thereof with a load of 45% w/w or more.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis—“TGA”, or differential scanning calorimetry—“DSC”), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, e.g., a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Rucaparib.

### SUMMARY OF THE DISCLOSURE

The present disclosure provides solid state forms of Rucaparib Tosylate, processes for preparation thereof, and pharmaceutical compositions thereof. These solid state forms can be used to prepare other forms of Rucaparib, Rucaparib salts and solid state forms thereof.

The present disclosure provides solid state forms of Rucaparib Tosylate for use in the preparation of pharmaceutical compositions and/or formulations for use in medicine, preferably for the treatment of cancer.

The present disclosure also encompasses the use of solid state forms of Rucaparib Tosylate of the present disclosure for the preparation of pharmaceutical compositions and/or formulations.

In another aspect, the present disclosure provides pharmaceutical compositions and/or pharmaceutical formulations comprising any one or a combination of the solid state forms of Rucaparib Tosylate according to the present disclosure.

In yet another embodiment, the present disclosure encompasses pharmaceutical formulations comprising any one or a combination of the described solid state forms of Rucaparib Tosylate, or pharmaceutical compositions comprising them and at least one pharmaceutically acceptable excipient.

The present disclosure comprises processes for preparing the above mentioned pharmaceutical formulations. The processes comprise combining any one of or a combination of the described solid state forms of Rucaparib Tosylate; or a pharmaceutical composition comprising them with at least one pharmaceutically acceptable excipient.

The solid state forms as defined herein and the pharmaceutical compositions or formulations of solid state forms of Rucaparib Tosylate may be used as medicaments, particularly for the treatment of cancer, for the treatment of patients with deleterious BRCA mutation (germline and/or somatic) associated advanced ovarian cancer who have been treated with two or more chemotherapies, or for the treatment of patients with prostate cancer, fallopian tube cancer and/or peritoneal cancer.

The present disclosure also provides methods of treating cancer comprising administering a therapeutically effective amount of any one or a combination of the solid state forms of Rucaparib Tosylate of the present disclosure, or at least one of the above pharmaceutical compositions or formulations, to a subject suffering from cancer, for the treatment of patients with deleterious BRCA mutation (germline and/or somatic) associated advanced ovarian cancer who have been treated with two or more chemotherapies, or for the treatment of patients with prostate cancer, fallopian tube cancer and/or peritoneal cancer, or otherwise in need of the treatment.

The present disclosure also provides the uses of solid state forms of Rucaparib Tosylate of the present disclosure, or at least one of the above pharmaceutical compositions or formulations, for the manufacture of medicaments for treating cancer. for the treatment of patients with deleterious BRCA mutation (germline and/or somatic) associated advanced ovarian cancer who have been treated with two or more chemotherapies, or for the treatment of patients with prostate cancer, fallopian tube cancer and/or peritoneal cancer.

The present disclosure also provides methods of treating cancer comprising administering a therapeutically effective amount of any one or a combination of the solid state forms of Rucaparib Tosylate of the present disclosure, or at least one of the above pharmaceutical compositions and/or formulations, to a subject in need of the treatment.

### BRIEF DESCRIPTION OF THE DRAWINGS

FIG. 1 shows a characteristic X-ray powder diffraction pattern (XRPD) of Rucaparib Tosylate Form X.

FIG. 2 shows a characteristic X-ray powder diffraction pattern of Rucaparib Tosylate Form III.

FIG. 3 shows a characteristic X-ray powder diffraction pattern of Rucaparib base Form C.

FIG. 4 shows a characteristic X-ray powder diffraction pattern of Rucaparib Tosylate Form X, obtained according to Example 3.

FIG. 5 shows a characteristic X-ray powder diffraction pattern of Rucaparib Tosylate Form X, obtained according to Example 4.

FIG. 6 shows a characteristic X-ray powder diffraction pattern of Rucaparib Tosylate Form XII.

FIG. 7 shows a characteristic X-ray powder diffraction pattern of Rucaparib Tosylate Form XIII.

FIG. 8 shows a characteristic X-ray powder diffraction pattern of Rucaparib Tosylate Form IX.

FIG. 9 shows PSD of Rucaparib Tosylate Form X, obtained according to Example 1.

FIG. 10 shows a SEM image of Rucaparib Tosylate Form X, obtained according to Example 1.

FIG. 11 shows PSD of Rucaparib Tosylate Form X, obtained according to Example 3.

FIG. 12 shows a SEM image of Rucaparib Tosylate Form X, obtained according to Example 3.

FIG. 13 shows a characteristic X-ray powder diffraction pattern of Rucaparib Tosylate Form X, obtained according to Example 8.

FIG. 14 shows a characteristic X-ray powder diffraction pattern of Rucaparib Tosylate Form X, obtained according to Example 9.

FIG. 15 shows PSD of Rucaparib Tosylate Form X, obtained according to Example 8.

FIG. 16 shows a characteristic X-ray powder diffraction pattern of Rucaparib Tosylate Form III, obtained according to Example 10.

FIG. 17 shows a characteristic X-ray powder diffraction pattern of Rucaparib Tosylate Form X, obtained according to Example 11.

FIG. 18 shows PSD of Rucaparib Tosylate Form X, obtained according to Example 11.

FIG. 19 shows a characteristic X-ray powder diffraction pattern of Rucaparib Tosylate Form X, obtained according to Example 13.

FIG. 20 shows PSD of Rucaparib Tosylate Form X, obtained according to Example 13.

FIG. 21 shows a characteristic X-ray powder diffraction pattern of Rucaparib Tosylate Form III, obtained according to Example 14.

FIG. 22 show a characteristic DSC thermogram of Rucaparib Tosylate Form X.

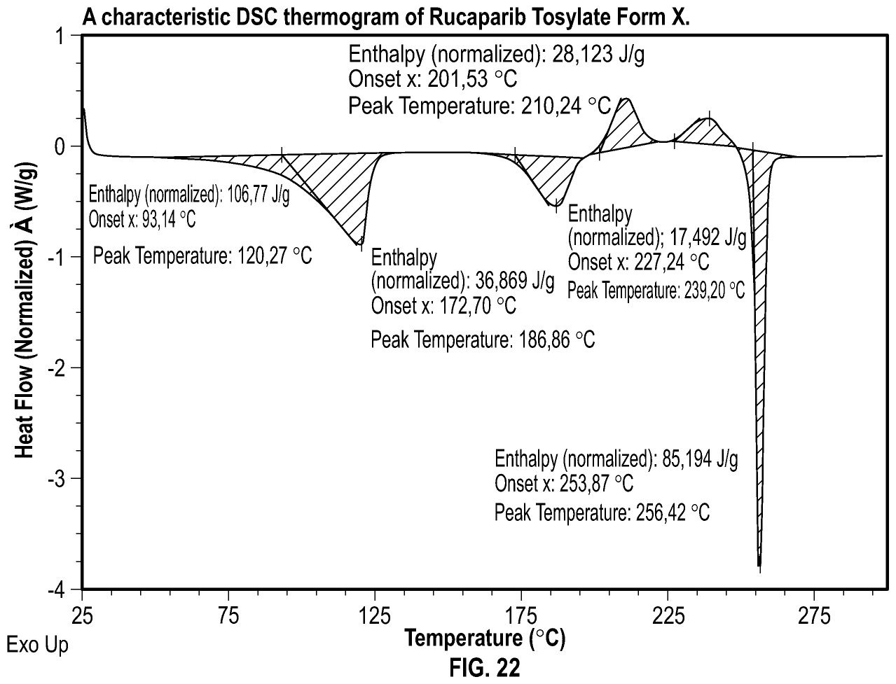

FIG. 23 shows a characteristic TGA thermogram of Rucaparib Tosylate Form X.

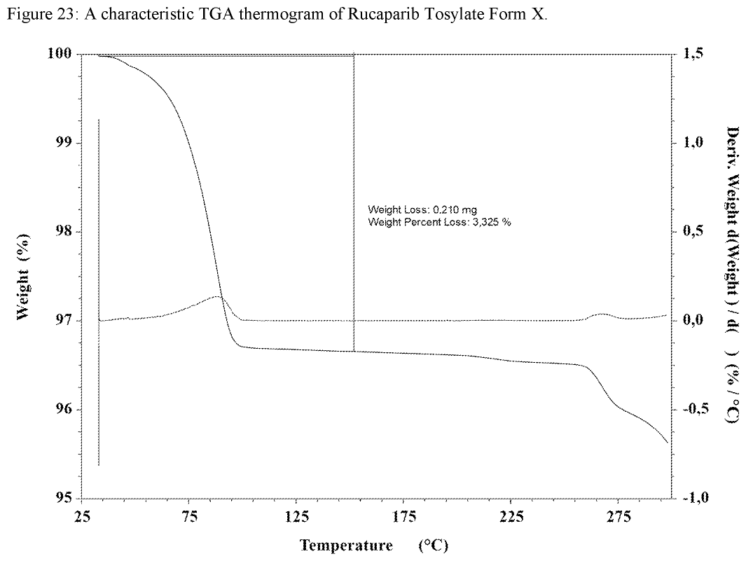

### DETAILED DESCRIPTION OF THE DISCLOSURE

The present disclosure encompasses solid state forms of Rucaparib Tosylate. Solid state properties of solid state forms of Rucaparib and salts thereof can be influenced by controlling the conditions under which the solid state forms of Rucaparib and of Rucaparib salts are obtained.

A solid state form (or polymorph) may be referred to herein as polymorphically pure or as substantially free of any other solid state (or polymorphic) forms. As used herein in this context, and unless indicated otherwise, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, solid state forms of Rucaparib and salts thereof described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject solid state form of Rucaparib or salt thereof. For example, Rucaparib tosylate described herein as being polymorphically pure or substantially free of any other solid state or polymorphic forms contains: about 20% (w/w) or less, about 10% (w/w) or less, about 5% (w/w) or less, about 2% (w/w) or less, about 1% (w/w) or less, or about 0% of any other forms of Rucaparib tosylate, as measured, for example, by XRPD. In some embodiments of the disclosure, the described solid state forms of Rucaparib or salts thereof may contain from about 1% to about 20% (w/w), from about 5% to about 20% (w/w), or from about 5% to about 10% (w/w) of one or more other solid state forms of the same Rucaparib or salt thereof.

A compound may be referred to herein as chemically pure or purified compound or as substantially free of any other compounds. As used herein in this context, the expression “substantially free of any other compounds” will be understood to mean that the pure compound contains about 20% (w/w) or less, about 10% (w/w) or less, about 5% (w/w) or less, about 2% (w/w) or less, about 1% (w/w) or less, or about 0% of any other compound as measured, for example, by HPLC. Thus, pure or purified Rucaparib or Rucaparib salt, such as Rucaparib tosylate, herein as substantially free of any compounds would be understood to contain greater than about 80% (w/w), greater than about 90% (w/w), greater than about 95% (w/w), greater than about 98% (w/w), greater than about 99% (w/w), or about 100% of the subject Rucaparib or Rucaparib salt. In some embodiments of the disclosure, the described pure or purified Rucaparib or Rucaparib salt, such as Rucaparib tosylate, may contain from about 1% to about 20% (w/w), from about 5% to about 20% (w/w), or from about 5% to about 10% (w/w) of one or more other compounds.

As used herein, crystalline Form III of Rucaparib Tosylate refers to Rucaparib Tosylate characterized as described in International Publication No. WO 2018/140377, the entire disclosure of which is incorporated herein by reference.

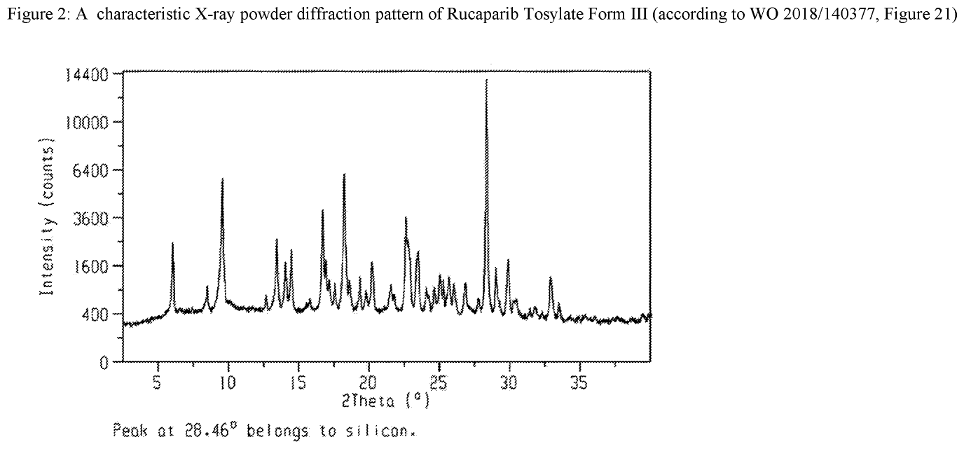

For example, crystalline Form III of Rucaparib Tosylate can be described by data selected from one or more of the following: an X-ray powder diffraction pattern substantially as depicted in FIG. 2; an X-ray powder diffraction pattern having peaks at 10.0, 13.8, 14.5, 17.0 and 18.5 degrees 2-theta±0.2 degrees 2-theta; and combinations of these data.

Crystalline Form III of Rucaparib Tosylate may be further characterized by an X-ray powder diffraction pattern having peaks at 10.0, 13.8, 14.5, 17.0 and 18.5 degrees 2-theta±0.2 degrees 2-theta, and also having any one, two, three, four or five additional peaks selected from the group consisting of 6.5, 20.4, 22.9, 23.7 and 25.8 degrees 2-theta±0.2 degrees 2-theta.

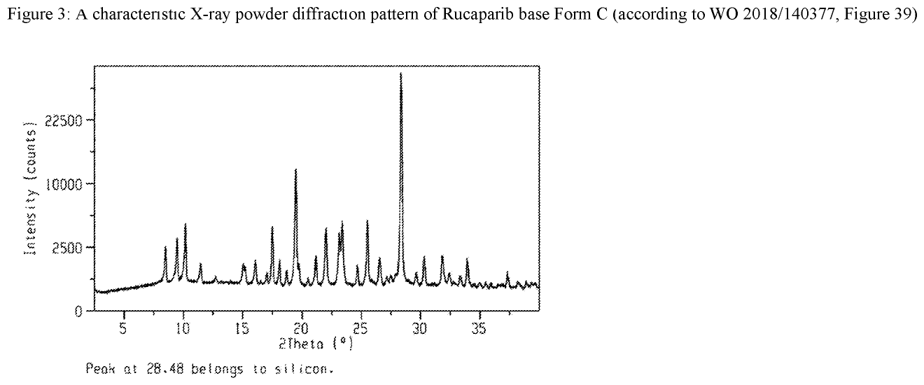

As used herein, crystalline Form C of Rucaparib base may be characterized as described in International Publication No. WO 2018/140377. Thus, as used herein, crystalline Form C of Rucaparib base refers to Rucaparib base characterized by an X-ray powder diffraction pattern having peaks at 10.5, 16.3, 19.7, 21.4, and 22.2 degrees 2-theta±0.2 degrees 2-theta. Alternatively crystalline Form C of Rucaparib base may refer to Rucaparib base characterized by an X-ray powder diffraction pattern having peaks at 10.5, 16.3, 19.7, 21.4, and 22.2 degrees 2-theta±0.2 degrees 2-theta, and also having any one, two, three, four or five additional peaks selected from the group consisting of 8.8, 15.5, 17.7, 18.4, and 26.7 degrees 2-theta±0.2 degrees 2-theta. Crystalline Form C or Rucaparib base may be alternatively characterized by an XRPD diffraction pattern substantially as depicted in FIG. 3.

Depending on which other solid state forms a comparison is made, the crystalline forms of Rucaparib and salts thereof of the present disclosure have advantageous properties selected from at least one of the following: chemical purity, flowability, solubility, dissolution rate, morphology or crystal habit, stability-such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility, and bulk density. Particularly the crystalline forms of Rucaparib Tosylate according to the present disclosure may have advantageous properties selected from one or more of stability under mechanical stress and solubility or kinetic solubility.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Rucaparib or salt thereof referred to herein as being characterized by graphical data “as depicted in” or “as substantially depicted in” a Figure will thus be understood to include any crystal forms of Rucaparib or salt thereof characterized with the graphical data having such small variations, as are well known to the skilled person, in comparison with the Figure.

As used herein, and unless stated otherwise, the term “anhydrous” in relation to crystalline forms of Rucaparib and salts thereof, relates to a crystalline form of Rucaparib or salt thereof which does not include any crystalline water (or other solvents) in a defined, stoichiometric amount within the crystal. Moreover, an “anhydrous” form would typically not contain more than 1% (w/w) of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to solid state forms of Rucaparib and salts thereof of the present disclosure corresponds to a solid state form of Rucaparib or salt thereof that is physically separated from the reaction mixture in which it is formed. The step of isolating solid state forms of Rucaparib or solid state forms of salts of Rucaparib may be performed by crystallization or precipitation.

As used herein, unless stated otherwise, the XRPD measurements are taken using copper Kα radiation wavelength 1.54184 Å. XRPD peaks reported herein are measured using Cuk α radiation, λ=1.5418 Å, at a temperature of 25±3° C.

As used herein, unless stated otherwise, ¹H, ¹³C and ¹⁵N NMR reported herein are measured at 400 MHZ, 100 MHz and 40 MHz, respectively, preferably at a temperature of at 293 K±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, typically about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The individual particles of a sample or aliquot of the solid particulate comprising crystalline Form X of Rucaparib tosylate of the present disclosure are not of uniform size. Rather, a sample or aliquot of a solid particulate comprising crystalline Form X of Rucaparib tosylate of the present disclosure is comprised of particles of different sizes that can be size-classified or distributed in an array of discrete, adjacent intervals of particle size. If the size of the intervals is small enough, the array of particle sizes approaches a continuum of particle sizes. This collection of discrete particle size intervals together with their population is referred to as the particle size distribution (PSD).

Measurement and characterization of particle size distributions is known in the art. It is possible to compare samples of particulate comprising crystalline Form X of Rucaparib tosylate on the basis of individual points on a cumulative particle size distribution curve. The measurements are represented as Dx(X)=Y (where X and Y are Arabic numerals), each “Dx” describing an individual point on a cumulative PSD curve. The number “X” represents the percentage (number, volume, or weight) of particles in the population having a nominal size up to and including “Y”. Thus, Dx(X)=250 μm is characteristic of a PSD in which 90% (number, volume, or weight) of the particles in a population have a nominal size of about 250μ or less (at least some particles having a nominal dimension of 250μ) and so forth. When PSD is determined by the well-known laser-diffraction method described herein, the Dx(X) measurement depicts a volume average.

The skilled artisan knows that the results of PSD determination by one technique can be correlated with results from another technique on an empirical basis by routine experimentation.

As used herein, particle size distribution is determined by means of laser diffractometry. More specifically, unless otherwise indicated, the particle size was determined using a Mastersizer 3000 from Malvern Instruments—the particle size determination may be carried out as a wet or dry measurement depending on the sample. Preferably, particle size and particle size distribution are determined by laser light diffraction, using Mie theory, and n-hexane as dispersing medium; more preferably, particle size and particle size distribution are determined by laser light diffraction using a Mastersizer 3000 from Malvern Instruments, using Mie theory, and n-hexane/0.2% dioctyl sulfosuccinate sodium salt as dispersing medium.

As used herein, unless otherwise indicated, “median particle size” refers to the D50 value of the particle size distribution.

The median particle size (D50, or Dx(50)), which is also denoted D50-value of the integral volume distribution, is defined herein as the particle diameter, at which 50 percent by volume of the particles have a smaller diameter than the diameter which corresponds to the D50-value. Likewise, 50 percent by volume of the particles have a larger diameter than the D50-value. Analogously, the D90-value (also referred to as Dx(90)) of the integral volume distribution is defined as the particle diameter, at which 90 percent by volume of the particles have a smaller diameter than the diameter, which corresponds to the D90-value. Correspondingly, the D10-value (also referred to as Dx(10)) of the integral volume distribution is defined as the particle diameter, at which 10 percent by volume of the particles have a smaller diameter than the diameter, which corresponds to the D10-value.

As used herein, “particle diameter” or “particle size” of a particle to be determined means the diameter of an equivalent particle which is assumed to be spherical and to have the same light scattering pattern as the particle to be determined.

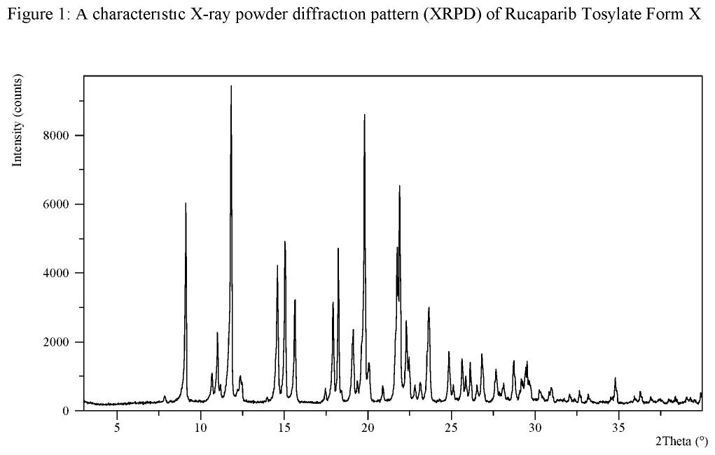

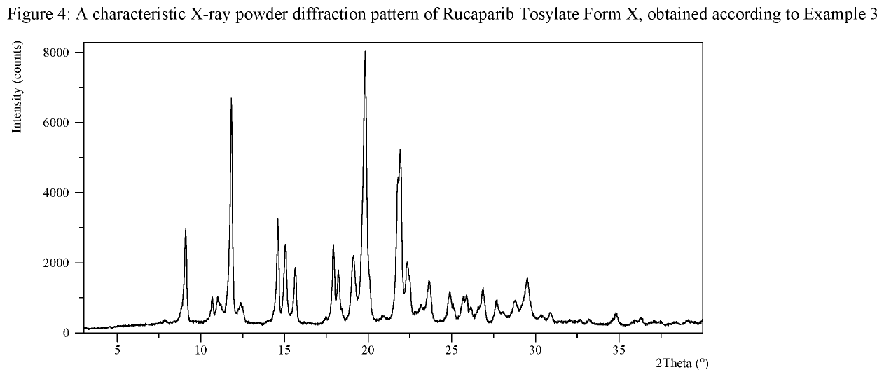

In one embodiment, the present disclosure relates to a crystalline form of Rucaparib Tosylate, designated Form X. The crystalline Form X of Rucaparib Tosylate may be characterized by data selected from one or more of the following: an X-ray powder diffraction pattern substantially as depicted in FIG. 1; an X-ray powder diffraction pattern having peaks at 9.1, 11.8, 15.6, 19.8 and 21.9 degrees 2-theta±0.2 degrees 2-theta; and combinations of these data. Crystalline Form X of Rucaparib tosylate may alternatively be characterized by an X-ray powder diffraction pattern substantially as depicted in any of FIGS. 4, 5, 13, 14, 17 and 19.

Crystalline Form X of Rucaparib Tosylate may be further characterized by an X-ray powder diffraction pattern having peaks at 9.1, 11.8, 15.6, 19.8 and 21.9 degrees 2-theta±0.2 degrees 2-theta and also having any one, two, three, four or five additional peaks selected from the group consisting of 10.7, 14.6, 17.9, 19.1 and 26.8 degrees 2-theta±0.2 degrees 2-theta.

Crystalline Form X of Rucaparib Tosylate may be alternatively characterized by an X-ray powder diffraction pattern having peaks at 9.1, 10.7, 11.8, 14.6, 15.6, 17.9, 19.1, 19.8, 21.9 and 26.8 degrees 2-theta±0.2 degrees 2-theta.

Crystalline Form X of Rucaparib Tosylate may be characterized by a XRPD pattern having peaks as listed in the below Table 1, ±0.2 degrees 2-theta. Optionally, Crystalline Form X of Rucaparib Tosylate may be characterized by an XRPD pattern having the 2-theta values in Table 1 together with an error value of ±0.2 degrees 2-theta, either with or without the respective relative intensity values)

TABLE 1

| Rel. Int. [%] | Pos. [°2Th.] |
|---|---|
| 9.1 | 62.12 |
| 10.7 | 9.41 |
| 11.0 | 22.08 |
| 11.2 | 5.75 |
| 11.8 | 100 |
| 12.4 | 8.46 |
| 12.5 | 6.09 |
| 14.6 | 42.28 |
| 15.1 | 49.44 |
| 15.6 | 33.4 |
| 17.5 | 4.44 |
| 17.9 | 31.47 |
| 18.2 | 45.76 |
| 19.1 | 22.03 |
| 19.4 | 6.87 |
| 19.6 | 17.21 |
| 19.8 | 91.32 |
| 20.1 | 12.25 |
| 20.9 | 5.37 |
| 21.6 | 17.36 |
| 21.7 | 47.4 |
| 21.9 | 67.8 |
| 22.3 | 25.24 |
| 22.5 | 14.37 |
| 22.8 | 5.49 |
| 23.1 | 6.36 |
| 23.7 | 29.72 |
| 24.9 | 15.98 |
| 25.1 | 5.42 |
| 25.6 | 13.64 |
| 25.9 | 8.18 |
| 26.1 | 12.15 |
| 26.5 | 5.69 |
| 26.8 | 15.37 |
| 27.7 | 10.68 |
| 28.1 | 6.22 |
| 28.7 | 12.84 |
| 29.2 | 7.23 |
| 29.4 | 10.28 |
| 29.5 | 13.35 |
| 29.7 | 5.72 |
| 30.2 | 4.01 |
| 30.8 | 3.42 |
| 31.0 | 4.94 |
| 34.8 | 7.87 |

Alternatively, crystalline Form AX2 of Rucaparib Tosylate may be characterized by the following unit cell data, which is as determined at a temperature of about 298 K:

| Unit cell parameter | A2, LeBail 298 K |
|---|---|
| Cell length a | 9.7503(3) Å |
| Cell length b | 15.8393(6) Å |
| Cell length c | 16.1027(5) Å |
| Cell angle alpha | 90° |
| Cell angle beta | 92.509(3)° |
| Cell angle gamma | 90° |
| Cell volume | 2484.48(14) Å3 |
| Z | 4 |
| Symmetry cell setting | monoclinic |
| Symmetry space group name_H_M | P21/c |

The R factor of the unit cell measurement is 8.40%.

Crystalline Form X of Rucaparib Tosylate may be characterized by each of the above characteristics alone/or by all possible combinations, e.g., an XRPD pattern having peaks at 9.1, 11.8, 15.6, 19.8 and 21.9 degrees 2-theta±0.2 degrees 2-theta; an XRPD pattern as depicted in FIG. 1, and combinations thereof.

Crystalline Form X of Rucaparib Tosylate may be a hydrate form. Typically, the weight loss by TGA is of about 0.5% to about 3.5%, or about 3.5% (w/w), and the water content by KF is of about 3.7%. Preferably, the water content of Form X is: about 2.0 wt % to about 5.0 wt %, about 2.5 wt % to about 4.5 wt %, about 3.0 to about 4.2 wt %, about 3.2 wt % to about 4.0 wt %, about 3.4 wt % to about 3.8 wt %, about 3.5 wt % to about 3.7 wt %, or about 3.7 wt %, preferably as measured by KF. Crystalline Form X may be a monohydrate.

In one embodiment of the present disclosure, crystalline Form X of Rucaparib Tosylate is isolated.

According to any embodiment of the present disclosure, crystalline Form X of Rucaparib Tosylate as described herein (i.e. by characteristic XRPD peaks and/or unit cell data) may be further characterized by having a PSD Dx(90) of: about 400 μm to about 960 μm, about 400 μm to about 950 μm, about 410 μm to about 940 μm, about 420 μm to about 930 μm, about 420 μm to about 920 μm, about 450 μm to about 910 μm, or from about 400 μm to about 910 μm, preferably of about 910 μm or about 450 μm. Optionally, according to any embodiment of the present disclosure, crystalline Form X of Rucaparib Tosylate as described herein (i.e. by characteristic XRPD peaks and/or unit cell data) may be alternatively further characterized by having a PSD Dx(90) of: about 700 μm to about 960 μm, about 750 μm to about 960 μm, about 770 μm to about 960 μm, about 800 μm to about 950 μm, about 850 to about 940 μm, about 880 μm to about 930 μm, about 890 μm about 920 μm, about 800 μm to about 910 μm, or from about 900 μm to about 910 μm or about 910 μm. Further optionally, according to any embodiment of the present disclosure, crystalline Form X of Rucaparib Tosylate as described herein (i.e. by characteristic XRPD peaks and/or unit cell data) may be alternatively further characterized by having a PSD Dx(90) of: about 400 μm to about 700 μm, about 410 μm to about 650 μm, about 420 μm to about 600 μm, about 430 μm to about 550 μm, about 440 μm to about 520 μm, about 440 μm to about 500 μm, about 450 μm to about 490 μm, about 400 μm to about 600 μm, or from about 450 μm to about 457 μm. Even further optionally, according to any embodiment of the present disclosure, crystalline Form X of Rucaparib Tosylate as described herein (i.e. by characteristic XRPD peaks and/or unit cell data) may be alternatively further characterized by having a PSD Dx(90) of: about 380 μm to about 600 μm, about 380 to about 580 μm, about 390 μm to about 560 μm, about 400 μm to about 550 μm, about 410 μm to about 520 μm, about 420 μm to about 500 μm, about 420 μm to about 460 μm, about 420 μm to about 440 μm, about 400 μm to about 600 μm, or from about 450 μm to about 460 μm, for example, about 455 μm or about 430 μm. Crystalline Form X of Rucaparib Tosylate having the PSD Dx(90) as described above, may be particularly useful for the preparation of crystalline Form II of Rucaparib tosylate. Crystalline Form X of Rucaparib Tosylate having the PSD Dx(90) described above, may be particularly useful for the preparation of crystalline Form III of Rucaparib tosylate.

According to another embodiment, crystalline Form X of Rucaparib Tosylate as described herein (i.e. by characteristic XRPD peaks and/or unit cell data), may be further characterized by a PSD Dx(90) of: about 90 μm to about 200 μm, about 100 μm to about 180 μm, about 100 μm to about 160 μm, about 110 μm to about 150 μm, about 120 μm to about 140 μm, about 120 μm to about 135 μm, or about 100 μm to about 129 μm, preferably about 129 μm.

Crystalline Form X of Rucaparib Tosylate can be used to prepare other solid state forms of Rucaparib Tosylate. Particularly, it can be used to prepare Crystalline Form III of Rucaparib Tosylate. Form X can also be used to prepare Crystalline Forms XII, XIII and/or IX of Rucaparib Tosylate.

In certain embodiment, the present disclosure encompasses a process for preparing Crystalline Form III of Rucaparib Tosylate comprising preparing Crystalline Form X of Rucaparib Tosylate by the process disclosed herein, and converting it to Crystalline Form III of Rucaparib Tosylate.

In particular, Crystalline Form X of Rucaparib Tosylate of certain particle size distribution (“PSD”) can be used to prepare Crystalline Form III of Rucaparib Tosylate. Typically, the mean PSD or PSD of Crystalline Form X of Rucaparib Tosylate used to prepare Crystalline Form III of Rucaparib Tosylate is of Dx(90) of from: about 400 μm to about 960 μm, about 400 μm to about 950 μm, about 410 μm to about 940 μm, about 420 μm to about 930 μm, about 420 μm to about 920 μm, about 450 μm to about 910 μm, or from about 400 μm to about 910 μm, preferably of about 910 μm or about 450 μm.

The present disclosure encompasses a process for preparing Crystalline Form III of Rucaparib Tosylate comprising suspending Rucaparib Tosylate Form X in 2-propanol.

Typically, the suspension of Rucaparib Tosylate Form X in 2-propanol is heated, for example to a temperature of: about 35° C. to about 80° C., about 40° C. to about 70° C., about 45° C. to about 60° C., about 45° C. to about 55° C., or about 50° C. Preferably, following the suspending in 2-propanol, the product is isolated at the elevated temperature, for example by filtration. The amount of 2-propanol may be from: about 2 ml to about 20 ml, about 5 ml to about 15 ml, about 8 ml to about 12 ml, or about 10 ml, per gram of Rucaparib Tosylate Form X. The suspension can be further maintained, for example for one hour or until a solid precipitates. Typically, it is maintained while stirring. Preferably, the solid is precipitated at the elevated temperature, i.e. at a temperature of about 35° C. to about 80° C., about 40° C. to about 70° C., about 45° C. to about 60° C., about 45° C. to about 55° C., or about 50° C. The precipitated solid can be isolated, for example by filtration or vacuum filtration.

Typically, the Rucaparib Tosylate Form X used in the above described process for preparing Rucaparib tosylate Form III, is having mean PSD or PSD as described above. Preferably, the mean PSD or PSD of Rucaparib Tosylate Form X in this process is from: about 700 μm to about 960 μm, about 750 μm to about 960 μm, about 770 μm to about 960 μm, about 800 μm to about 950 μm, about 850 to about 940 μm, about 880 μm to about 930 μm, about 890 μm about 920 μm, about 800 μm to about 910 μm, or from about 900 μm to about 910 μm, or about 910 μm.

Alternatively, Crystalline Form III of Rucaparib Tosylate can be prepared by a process comprising suspending Rucaparib Tosylate Form X in acetone. Typically, the suspension of Rucaparib Tosylate Form X in acetone is heated, for example to a temperature of; about 35° C. to about 80° C., about 40° C. to about 70° C., about 45° C. to about 60° C., about 45° C. to about 55° C., or about 50° C. The amount of acetone may be from: about 2 ml to about 20 ml, about 5 ml to about 15 ml, about 8 ml to about 12 ml, or about 10 ml, per gram of Rucaparib Tosylate Form X. The suspension can be further maintained, for example for: about 4 hours to about 24 hours, about 6 hours to about 20 hours, about 8 hours to about 18 hours, about 10 hours to about 14 hours, or about 12 hours. The suspension can be cooled, for example, to a temperature such as room temperature, and can be maintained until a solid precipitates, typically over a period of about 10 minutes to about 2 hours, about 10 minutes to about 1.5 hours, about 10 minutes to about 1 hour, about 15 minutes to about 45 minutes, or about 30 minutes. The cooling may be conducted at a rate of about 0.5° C. to about 2° C. per minute, about 0.5° C. to about 1.5° C. per minute, about 0.6° C. to about 1.2° C. per minute, about 0.8° C. to about 1.0° C. per minute, or about 0.9° C. per minute. Typically, it is maintained at the cooled temperature (preferably room temperature). Typically, it is maintained while stirring. The precipitated solid can be isolated, for example by filtration or vacuum filtration. The isolated solid may be dried, for example by vacuum drying, at a temperature of about 80° C.

Typically, the Rucaparib Tosylate Form X used in the above described process for preparing Rucaparib tosylate Form III, is having mean PSD or PSD as described above. In embodiments, it has PSD of Dx(90) of from about 400 μm to about 800 μm, for example, or from: about 400 μm to about 700 μm, about 410 μm to about 650 μm, about 420 μm to about 600 μm, about 430 μm to about 550 μm, about 440 μm to about 520 μm, about 440 μm to about 500 μm, about 450 μm to about 490 μm, about 400 μm to about 600 μm, or from about 450 μm to about 457 μm.

Another process for preparing Crystalline Form III of Rucaparib Tosylate according to the present disclosure comprises suspending Rucaparib Tosylate Form X in acetonitrile. Typically, the suspension of Rucaparib Tosylate Form X in acetonitrile is heated, for example to a temperature of: about 45° C. to about 80° C., about 50° C. to about 80° C., about 60° C. to about 80° C., about 65° C. to about 80° C., about 70° C. to about 80° C. or about 75° C. The amount of acetonitrile may be from: about 2 ml to about 20 ml, about 5 ml to about 15 ml, about 8 ml to about 12 ml, or about 10 ml, per gram of Rucaparib Tosylate Form X. The suspension can be further maintained, for example for: about 30 minutes to about 16 hours, about 1 hour to about 14 hours, about 2 hours to about 10 hours, about 3 hours to about 9 hours, about 3 hours to about 8 hours, or about 4 hours, or about 8 hours. The suspension can be cooled, for example, to a temperature such as room temperature or about 22° C., and can be maintained until a solid precipitates, typically, over a period of: about 10 minutes to about 4 hours, about 10 minutes to about 2 hours, about 10 minutes to about 1.5 hours, about 10 minutes to about 1 hour, about 15 minutes to about 45 minutes, or about 30 minutes. The cooling may be conducted at a rate of about 0.5° C. to about 3° C. per minute, about 1.0° C. to about 2.5° C. per minute, about 1.2° C. to about 2.2° C. per minute, about 1.5° C. to about 2.0° C. per minute, or about 1.7° C. per minute. Typically, it is maintained at the cooled temperature (preferably room temperature), while stirring. The precipitated solid can be isolated, for example by filtration or vacuum filtration. The isolated solid may be dried, for example by vacuum drying, at a temperature of about 80° C.

Typically, the Rucaparib Tosylate Form X used in the above described process for preparing Rucaparib tosylate Form III, is having mean PSD or PSD as described above. In embodiments, it has PSD of Dx(90) of from: about 400 μm to about 600 μm, about 400 to about 580 μm, about 400 μm to about 560 μm, about 400 μm to about 550 μm, about 410 μm to about 520 μm, about 420 μm to about 500 μm, about 420 μm to about 460 μm, about 420 μm to about 440 μm, about 400 μm to about 600 μm, or from about 450 μm to about 460 μm, for example, about 455 μm or about 430 μm.

The above-described processes for preparing Crystalline Form III of Rucaparib Tosylate may further comprise combining the Rucaparib Tosylate form III with at least one pharmaceutically acceptable excipient to prepare a pharmaceutical composition.

In another embodiment, the present disclosure encompasses a process for preparing Crystalline Form XII of Rucaparib Tosylate comprising preparing Crystalline Form X of Rucaparib Tosylate by the process disclosed herein, and converting it to Crystalline Form XII of Rucaparib Tosylate.

In particular, Crystalline Form X of Rucaparib Tosylate of certain particle size distribution (“PSD”) can be used to prepare Crystalline Form XII of Rucaparib Tosylate. Typically, the mean PSD or PSD of Crystalline Form X of Rucaparib Tosylate used to prepare Crystalline Form XII of Rucaparib Tosylate is of Dx(90) of from: about 90 μm to about 200 μm, about 100 μm to about 180 μm, about 100 μm to about 160 μm, about 110 μm to about 150 μm, about 120 μm to about 140 μm, about 120 μm to about 135 μm, or about 100 μm to about 129 μm, preferably about 129 μm.

The present disclosure encompasses a process for preparing Crystalline Form XII of Rucaparib Tosylate comprising suspending Rucaparib Tosylate Form X in 2-propanol.

Typically, the suspension of Rucaparib Tosylate Form X in 2-propanol is heated, for example to a temperature of: about 45° C. to about 90° C., about 50° C. to about 85° C., about 60° C. to about 80° C., about 65° C. to about 80° C., about 70° C. to about 80° C. or about 75° C. Preferably, following the suspending in 2-propanol, the product is isolated after cooling to room temperature, for example by filtration. The amount of 2-propanol may be from: about 2 ml to about 20 ml, about 5 ml to about 15 ml per gram of Rucaparib Tosylate Form X. The heating may be carried out for a period of: about 30 minutes to about 10 hours, about 1 hour to about 8 hours, about 2 hours to about 6 hours, about 3 hours to about 5 hours, or about 4 hours. The suspension may then be cooled, preferably by allowing the temperature to decrease to room temperature. The suspension can then be maintained, optionally upon stirring, for a period of: about 4 hours to about 48 hours, about 10 hours to about 35 hours, about 16 hours to about 30 hours, or about 24 hours or until a solid precipitates. Preferably, the suspension is precipitated at the cooled temperature, i.e. at about room temperature. The precipitated solid can be isolated, for example by filtration or vacuum filtration.

Typically, the Rucaparib Tosylate Form X used in the above described process for preparing Rucaparib Tosylate Form XII is having mean PSD or PSD as described above.

The above-described processes for preparing Crystalline Form XII of Rucaparib Tosylate may further comprise combining the Rucaparib Tosylate form XII with at least one pharmaceutically acceptable excipient to prepare a pharmaceutical composition.

The present disclosure also encompasses a process for preparing Crystalline Form XIII of Rucaparib Tosylate comprising heating Rucaparib Tosylate Form X to a temperature of: about 220° C. to about 255° C., about 230° C. to about 253° C., about 235° C. to about 252° C., about 240° C. to about 260° C., or about 250° C. and cooling to room temperature. Typically, the heating is performed at a heating rate of about 10° C./minute. When temperature of: about 220° C. to about 255° C., about 230° C. to about 253° C., about 235° C. to about 252° C., about 240° C. to about 260° C., or about 250° C. is reached, typically the sample is isothermally heated at this temperature for additional time, i.e., maintained at this temperature, for example for a period of: about 5 minutes to about 30 minutes, about 10 minutes to about 20 minutes, or about 15 minutes.

The present disclosure further encompasses a process for preparing Crystalline Form IX of Rucaparib Tosylate comprising suspending Rucaparib Tosylate Form X in toluene.

Typically, the suspension of Rucaparib Tosylate Form X in toluene is heated, for example to a temperature of: about 80° C. to about 130° C., about 90° C. to about 125° C., about 100° C. to about 120° C., about 105° C. to about 115° C., or about 110° C. The amount of toluene may be from: about 5 ml to about 20 ml, about 7 ml to about 15 ml, about 8 ml to about 12 ml, or about 10 ml, per gram of Rucaparib Tosylate Form X. The suspension may then be cooled, preferably by allowing the temperature to decrease to room temperature. The suspension can then be maintained, optionally upon stirring, until a solid precipitates. The precipitated solid can be isolated, for example by filtration or vacuum filtration.

Typically, the Rucaparib Tosylate Form X used in the above described process for preparing Rucaparib tosylate Form IX has a mean PSD or PSD of from: about 800 μm to about 960 μm, about 820 μm to about 960 μm, about 840 μm to about 950 μm, about 880 μm to about 940 μm, about 900 to about 940 μm, about 910 μm to about 940 μm, about 920 μm about 940 μm, or about 932 μm.

The above-described processes for preparing Crystalline Form IX of Rucaparib Tosylate may further comprise combining the Rucaparib Tosylate Form IX with at least one pharmaceutically acceptable excipient to prepare a pharmaceutical composition.

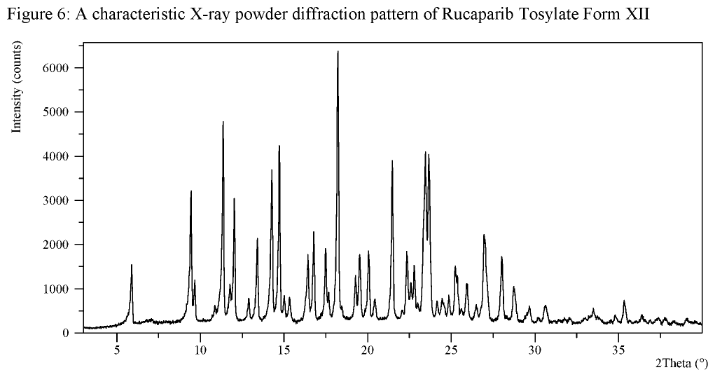

In one embodiment, the present disclosure relates to a crystalline form of Rucaparib Tosylate, designated Form XII. The crystalline Form XII of Rucaparib Tosylate may be characterized by data selected from one or more of the following: an X-ray powder diffraction pattern substantially as depicted in FIG. 6; an X-ray powder diffraction pattern having peaks at 9.4, 11.4, 13.4, 14.3 and 21.5 degrees 2-theta±0.2 degrees 2-theta; and combinations of these data.

Crystalline Form XII of Rucaparib Tosylate may be further characterized by an X-ray powder diffraction pattern having peaks at 9.4, 11.4, 13.4, 14.3 and 21.5 degrees 2-theta±0.2 degrees 2-theta and also having any one, two, three, four or five additional peaks selected from the group consisting of 5.9, 12.0, 16.4, 16.8 and 23.4 degrees 2-theta=0.2 degrees 2-theta.

Crystalline Form XII of Rucaparib Tosylate may be alternatively characterized by an X-ray powder diffraction pattern having peaks at 5.9, 9.4, 11.4, 12.0 13.4, 14.3, 16.4, 16.8, 21.5 and 23.4 degrees 2-theta±0.2 degrees 2-theta.

Crystalline Form XII of Rucaparib Tosylate may be characterized by a XRPD pattern having peaks as listed in the below Table 2, ±0.2 degrees 2-theta. Optionally, Crystalline Form XII of Rucaparib Tosylate may be characterized by an XRPD pattern having the 2-theta values in Table 2 together with an error value of ±0.2 degrees 2-theta, either with or without the respective relative intensity values):

TABLE 2

| Rel. Int. [%] | Pos. [°2Th.] |
|---|---|
| 5.9 | 22.1 |
| 9.4 | 48.27 |
| 9.7 | 16.89 |
| 10.9 | 7.44 |
| 11.4 | 73.56 |
| 11.8 | 15.66 |
| 12.0 | 46.36 |
| 12.9 | 10.31 |
| 13.4 | 30.71 |
| 14.3 | 55.67 |
| 14.7 | 66.56 |
| 15.0 | 11.21 |
| 15.3 | 10.56 |
| 16.4 | 25.49 |
| 16.8 | 35.07 |
| 17.5 | 28.17 |
| 17.7 | 12.52 |
| 18.2 | 100 |
| 18.5 | 7.39 |
| 19.3 | 17.52 |
| 19.5 | 26.08 |
| 20.1 | 26.9 |
| 20.4 | 10.14 |
| 21.5 | 59.87 |
| 22.1 | 5.83 |
| 22.3 | 26.87 |
| 22.6 | 16.3 |
| 22.8 | 22.76 |
| 23.0 | 8.77 |
| 23.3 | 34.56 |
| 23.4 | 63.27 |
| 23.7 | 63.17 |
| 24.1 | 9.15 |
| 24.4 | 8.99 |
| 24.8 | 11.33 |
| 25.2 | 21.79 |
| 25.4 | 18.68 |
| 25.6 | 6.46 |
| 25.9 | 15.78 |
| 26.5 | 8.05 |
| 26.9 | 32.42 |
| 27.0 | 30.37 |
| 28.0 | 25.68 |
| 28.7 | 14.06 |
| 29.7 | 7.25 |
| 30.6 | 6.6 |
| 33.5 | 6.67 |
| 35.3 | 9.5 |

Crystalline Form XII of Rucaparib Tosylate may be characterized by each of the above characteristics alone/or by all possible combinations, e.g., an XRPD pattern having peaks at 9.4, 11.4, 13.4, 14.3 and 21.5 degrees 2-theta±0.2 degrees 2-theta; an XRPD pattern as depicted in FIG. 6, and combinations thereof.

Crystalline Form XII of Rucaparib Tosylate may be a 2-propanol solvate form. The solvent content may be of about 6.6% (w/w), as analyzed by GC.

In one embodiment of the present disclosure, crystalline Form XII of Rucaparib Tosylate is isolated.

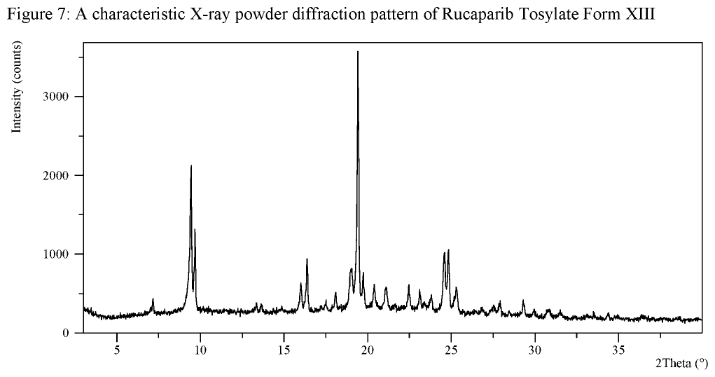

In one embodiment, the present disclosure relates to a crystalline form of Rucaparib Tosylate, designated Form XIII. The crystalline Form XIII of Rucaparib Tosylate may be characterized by data selected from one or more of the following: an X-ray powder diffraction pattern substantially as depicted in FIG. 7; an X-ray powder diffraction pattern having peaks at 9.7, 19.1, 19.4, 24.6 and 24.8 degrees 2-theta±0.2 degrees 2-theta; and combinations of these data.

Crystalline Form XIII of Rucaparib Tosylate may be further characterized by an X-ray powder diffraction pattern having peaks at 9.7, 19.1, 19.4, 24.6 and 24.8 degrees 2-theta±0.2 degrees 2-theta and also having any one, two, three or four additional peaks selected from the group consisting of 9.4, 16.0, 16.4 and 21.1 degrees 2-theta±0.2 degrees 2-theta.

Crystalline Form XIII of Rucaparib Tosylate may be alternatively characterized by an X-ray powder diffraction pattern having peaks at 9.4, 9.7, 16.0, 16.4, 19.1, 19.4, 21.1, 24.6 and 24.8 degrees 2-theta±0.2 degrees 2-theta.

Crystalline Form XIII of Rucaparib Tosylate may be characterized by a XRPD pattern having peaks as listed in the below Table 3, ±0.2 degrees 2-theta. Optionally, Crystalline Form XIII of Rucaparib Tosylate may be characterized by an XRPD pattern having the 2-theta values in Table 3 or Table 3A together with an error value of ±0.2 degrees 2-theta, either with or without the respective relative intensity values):

TABLE 3

| Rel. Int. [%] | Pos. [°2Th.] |
|---|---|
| 7.2 | 5.59 |
| 9.4 | 55.53 |
| 9.7 | 30.44 |
| 13.4 | 3.59 |
| 13.7 | 3 |
| 14.8 | 1.58 |
| 16.0 | 11.58 |
| 16.4 | 20.08 |
| 17.5 | 4.18 |
| 18.1 | 8.01 |
| 19.0 | 16.48 |
| 19.1 | 16.17 |
| 19.4 | 100 |
| 19.7 | 15.93 |
| 20.4 | 11.78 |
| 21.1 | 10.14 |
| 21.6 | 3.49 |
| 22.5 | 11.67 |
| 23.1 | 9.96 |
| 23.8 | 7.78 |
| 24.6 | 25.11 |
| 24.8 | 25.99 |
| 25.3 | 10.53 |
| 26.8 | 2.69 |
| 27.6 | 3.59 |
| 27.9 | 4.7 |
| 28.5 | 1.92 |
| 29.3 | 6.71 |
| 29.9 | 2.71 |
| 30.8 | 2.88 |
| 31.5 | 2.13 |
| 33.5 | 1.27 |
| 34.4 | 1.77 |
| 34.8 | 0.45 |
| 36.4 | 0.97 |
| 38.6 | 0.46 |

TABLE 3A

| Rel. Int. [%] | Pos. [°2Th.] |
|---|---|
| 7.2 | 5.59 |
| 9.4 | 55.53 |
| 9.7 | 30.44 |
| 13.4 | 3.59 |
| 13.7 | 3 |
| 16.0 | 11.58 |
| 16.4 | 20.08 |
| 17.5 | 4.18 |
| 18.1 | 8.01 |
| 19.0 | 16.48 |
| 19.1 | 16.17 |
| 19.4 | 100 |
| 19.7 | 15.93 |
| 20.4 | 11.78 |
| 21.1 | 10.14 |
| 21.6 | 3.49 |
| 22.5 | 11.67 |
| 23.1 | 9.96 |
| 23.8 | 7.78 |
| 24.6 | 25.11 |
| 24.8 | 25.99 |
| 25.3 | 10.53 |
| 27.6 | 3.59 |
| 27.9 | 4.7 |
| 29.3 | 6.71 |

Crystalline Form XIII of Rucaparib Tosylate may be characterized by each of the above characteristics alone/or by all possible combinations, e.g., an XRPD pattern having peaks at 9.7, 19.1, 19.4, 24.6 and 24.8 degrees 2-theta±0.2 degrees 2-theta; an XRPD pattern as depicted in FIG. 7, and combinations thereof.

Crystalline Form XIII of Rucaparib Tosylate may be an anhydrous form.

In one embodiment of the present disclosure, crystalline Form XIII of Rucaparib Tosylate is isolated.

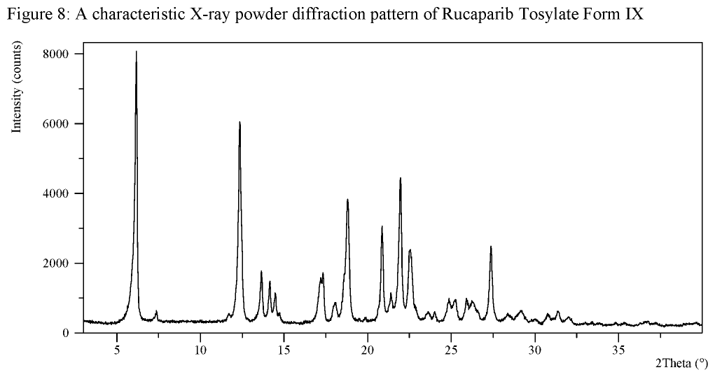

In one embodiment, the present disclosure relates to a crystalline form of Rucaparib Tosylate, designated Form IX. The crystalline Form IX of Rucaparib Tosylate may be characterized by data selected from one or more of the following: an X-ray powder diffraction pattern substantially as depicted in FIG. 8; an X-ray powder diffraction pattern having peaks at 6.2, 12.4, 18.8 and 20.9 degrees 2-theta±0.2 degrees 2-theta; and combinations of these data.

Crystalline Form IX of Rucaparib Tosylate may be further characterized by an X-ray powder diffraction pattern having peaks at 6.2, 12.4, 18.8 and 20.9 degrees 2-theta±0.2 degrees 2-theta and also having any one, two, three or four additional peaks selected from the group consisting of 13.7, 14.5, 22.5 and 27.4 degrees 2-theta±0.2 degrees 2-theta.

Crystalline Form IX of Rucaparib Tosylate may be alternatively characterized by an X-ray powder diffraction pattern having peaks at 6.2, 12.4, 13.7, 14.5, 18.8, 20.9, 22.5 and 27.4 degrees 2-theta±0.2 degrees 2-theta.

Crystalline Form IX of Rucaparib Tosylate may be characterized by a XRPD pattern having peaks as listed in the below Table 4, ±0.2 degrees 2-theta. Optionally, Crystalline Form IX of Rucaparib Tosylate may be characterized by an XRPD pattern having the 2-theta values in Table 4 together with an error value of ±0.2 degrees 2-theta, either with or without the respective relative intensity values)

TABLE 4

| Rel. Int. [%] | Pos. [°2Th] |
|---|---|
| 6.2 | 100 |
| 7.4 | 4.02 |
| 11.7 | 3 |
| 12.4 | 74.22 |
| 12.5 | 30.03 |
| 13.7 | 19.06 |
| 14.2 | 15.05 |
| 14.5 | 10.73 |
| 14.7 | 3.73 |
| 17.2 | 15.16 |
| 17.3 | 18.45 |
| 18.1 | 7.54 |
| 18.6 | 15.75 |
| 18.8 | 44.93 |
| 20.9 | 36.13 |
| 21.4 | 11.34 |
| 22.0 | 53.77 |
| 22.5 | 26.39 |
| 22.6 | 24.45 |
| 23.6 | 4.28 |
| 24.0 | 4.45 |
| 24.8 | 8.46 |
| 25.3 | 8.98 |
| 25.9 | 8.81 |
| 26.3 | 8.24 |
| 26.6 | 4.34 |
| 27.4 | 28.99 |
| 28.4 | 3.82 |
| 29.2 | 5.07 |
| 30.8 | 4.04 |
| 31.4 | 4.86 |

Crystalline Form IX of Rucaparib Tosylate may be characterized by each of the above characteristics alone/or by all possible combinations, e.g., an XRPD pattern having peaks at 6.2, 12.4, 18.8 and 20.9 degrees 2-theta±0.2 degrees 2-theta; an XRPD pattern as depicted in FIG. 8, and combinations thereof.

Crystalline Form IX of Rucaparib Tosylate may be a hydrate form.

In one embodiment of the present disclosure, crystalline Form IX of Rucaparib Tosylate is isolated.

The above-described solid state forms of Rucaparib Tosylate can be used to prepare other solid state forms of Rucaparib, Rucaparib tosylate and other Rucaparib salts and their solid state forms thereof.

In any aspect or embodiment of the present disclosure, any of the solid state forms of Rucaparib Tosylate described herein may be polymorphically pure or may be substantially free of any other solid state forms of Rucaparib Tosylate. In any aspect or embodiment of the present disclosure, any of the solid state forms of Rucaparib Tosylate may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject compound, preferably as measured by XRPD. Thus, any of the disclosed crystalline forms of Rucaparib Tosylate described herein may be substantially free of any other solid state forms of Rucaparib Tosylate, and may contain greater than about 80% (w/w), greater than about 90% (w/w), greater than about 95% (w/w), greater than about 98% (w/w), greater than about 99% (w/w), greater than about 99.5% (w/w), greater than about 99.8 (w/w) or about 100% of the subject crystalline form of Rucaparib Tosylate.

The above solid state forms of Rucaparib Tosylate can be used to prepare other solid state forms of Rucaparib or other solid state forms of salts of Rucaparib, such as other solid state forms of Rucaparib Tosylate or Rucaparib camsylate. Particularly, the present disclosure encompasses the use of Rucaparib Tosylate Form X for the preparation of other solid state forms of Rucaparib tosylate, preferably Rucaparib Tosylate Form III, Rucaparib Tosylate Form XII and Rucaparib Tosylate Form IX.

The present disclosure comprises a process for preparing solid state forms of Rucaparib and of Rucaparib salts, comprising preparing any one or a combination of the solid state forms of Rucaparib Tosylate of the present disclosure and converting it to another solid state form of Rucaparib or of Rucaparib salt.

The present disclosure provides solid state forms of Rucaparib Tosylate for use in the preparation of pharmaceutical compositions and/or formulations comprising solid state forms of Rucaparib and/or solid state forms of salts of Rucaparib.

The present disclosure also encompasses the use of the solid state forms of Rucaparib Tosylate of the present disclosure for the preparation of pharmaceutical compositions and/or formulations of solid state forms of Rucaparib Tosylate.

Pharmaceutical combinations or formulations of the present disclosure contain any one or a combination of the solid state forms of Rucaparib Tosylate of the present disclosure. In addition to the active ingredient, the pharmaceutical formulations of the present disclosure can contain one or more excipients. Excipients are added to the formulation for a variety of purposes.

In any aspect or embodiment of the disclosure, pharmaceutical compositions or formulations of the present disclosure include solid dosage forms such as tablets, powders, capsules, suppositories, sachets, troches, and lozenges, or liquid dosage forms such as liquid syrups, suspensions, and elixirs. In any aspect or embodiment of the disclosure, the pharmaceutical compositions or formulations of the present disclosure are solid dosage forms such as tablets, powders, capsules, suppositories, sachets, troches, and lozenges. Particularly, the pharmaceutical compositions or formulations of the present disclosure may be for oral administration, more particularly, the pharmaceutical compositions or formulations of the present disclosure may be tablets, powders, capsules, or lozenges, and especially tablets or capsules and more particularly tablets.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, Rucaparib and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Rucaparib can be administered. Rucaparib may be formulated for administration to a mammal, in embodiments to a human, by injection. Rucaparib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The present disclosure comprises processes for preparing the above mentioned pharmaceutical formulations. The processes comprise combining any one or a combination of the solid state forms of Rucaparib Tosylate of the present disclosure with at least one pharmaceutically acceptable excipient.

The solid state forms of Rucaparib Tosylate and the pharmaceutical compositions of solid state forms of Rucaparib Tosylate of the present disclosure, can be used as medicaments, particularly for the treatment of cancer, particularly for the treatment of patients with deleterious BRCA mutation (germline and/or somatic) associated advanced ovarian cancer who have been treated with two or more chemotherapies, or for the treatment of patients with prostate cancer, fallopian tube cancer and/or peritoneal cancer.

The present disclosure also provides methods of treating cancer comprising administering a therapeutically effective amount of any one or a combination of the solid state forms of Rucaparib Tosylate of the present disclosure, or at least one of the above pharmaceutical compositions, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”)

A sample is powdered in a mortar and pestle and applied directly on a silicon plate holder. The X-ray powder diffraction pattern was measured with Philips X'Pert PRO X-ray powder diffractometer, equipped with Cu irradiation source=1.54184 \acute{Å} (\acute{Å}ngström), X'Celerator (2.022° 2θ) detector. Scanning parameters: angle range: 3-40 deg., step size 0.0167, time per step 37 s, continuous scan.

The described peak positions were determined with or without using silicon powder as an internal standard in an admixture with the sample measured.

### Scanning Electron Microscope (“SEM”) Method:

Samples were glued to an aluminum holder with a double sided carbon tape.

Sputtered with gold by Edwards S150b gold coater. Current: 2 mA, time of coating: 5-10 minutes.

Micrographs were captured by Jeol JSM-5800 scanning microscope (FA1015235) under working distance of 20 mm and high tension of 7-10 kV.

### Particle Size Distribution (“PSD”) Method:

Instrument for laser diffraction: Malvern Mastersizer 3000.

Dispersion unit: Hydro MV. Calculation using Mie theory.

Sample is prepared as concentrated suspension.

Sample may be divided into representative samples by sample divider (riffler, if available). Sample preparation media: 0.2% of dioctyl sulfosuccinate sodium salt (DSSS) in n-hexane, measurement media: n-hexane. Number of measurement cycles: 5 (the term “mean PSD” used throughout therefore relates to the average PSD obtained from the 5 measurement cycles, and equates to the measured PSD of the sample, i.e. the term “mean PSD” is the PSD of the sample).

### Single Crystal Data Method:

A suitable crystal was selected and mounted directly on the goniometer of Xcalibur, Sapphire3, Gemini diffractometer. The crystal was kept at 298 K during data collection. Data collection was carried out using CrysAlis Pro (Rigaku Oxford Diffraction). Using Olex2 [1], the structure was solved with the SHELXT[2] structure solution program using Intrinsic Phasing and refined with the SHELXL[3] refinement package using Least Squares minimisation.

### EXAMPLES

Starting material may be prepared according to the disclosure of U.S. Patent Publication No. 2019/0389871.

Rucaparib can be prepared according to methods known from the literature, for example U.S. Pat. No. 6,495,541. A process for its preparation is described in U.S. Pat. No. 7,323,562, as well as in Org. Process Res. Dev., 2012, 16 (12), pp 1897-1904.

Rucaparib Form C can be prepared according to any one of the examples described in International Publication No. WO 2018/140377.

Alternatively, Rucaparib Form C can be prepared as follows:

Rucaparib Hydrochloride (19.41 grams) was suspended in methanol (90 ml). The reaction mixture was heated to a temperature of about 67° C. and the pH was adjusted to 11.5 by addition of aqueous NaOH (2M) over a period of 120 minutes. The obtained reaction mixture was further stirred for 1 hour at a temperature of about 65° C. to about 70° C. Then, water was added dropwise, in a volume to complete a total volume of water and aqueous NaOH-90 ml). The obtained suspension was cooled to a temperature of about 20° C. to 25° C. over a period of about 2 hours. The cooled suspension was filtered off and washed with 2×18 ml of methanol/water mixture (1/1 v/v) and then a third wash by 18 ml of water. The isolated material was dried at a temperature of about 50° C. until constant mass. A sample was and analyzed by XRPD, Form C was obtained.

Rucaparib Tosylate Form III can be prepared according to any one of the examples described in International Publication NO. WO 2018/140377.

For example, Rucaparib Tosylate Form III can be prepared according to Example 47A of International Publication NO. WO 2018/140377

To a suspension of 3.0 grams of Rucaparib Base in 90.0 ml of techn. EtOH at 20-25° C., 1.76 grams of p-toluenesulfonic acid monohydrate was added. After stirring for about 24 hours at 20-25° C. crystals were filtered off, washed twice with 7.5 ml of techn. EtOH and dried at 50° C./20 mbar for about 16 hours. 3.7 grams of Rucaparib Tosylate Form III was obtained.

### Example 1. Preparation of Rucaparib Tosylate Form X

Rucaparib free base (45 grams, Form C) was suspended in a solvent mixture of ethanol and water (625 ml, ratio 80:20 v/v) at room temperature for 25 minutes. p-toluenesulfonic acid monohydrate (26.9 grams, 1.02 eq.) was added to the suspension and heated to 80° C. to obtain a solution. The solution was additionally mixed for 10 minutes and cooled down to 53° C. Rucaparib Tosylate Form III (0.9 grams) was added to the solution and it was mixed for 30 minutes at 53° C. and then cooled to 2° C. over a period of about 3.5 hours. The sample was washed with 2×45 ml of ethanol, 96% and vacuum dried at 80° C. to a constant mass. The obtained solid was analyzed by XRPD and Rucaparib Tosylate Form X was obtained. XRPD pattern is shown in FIG. 1.

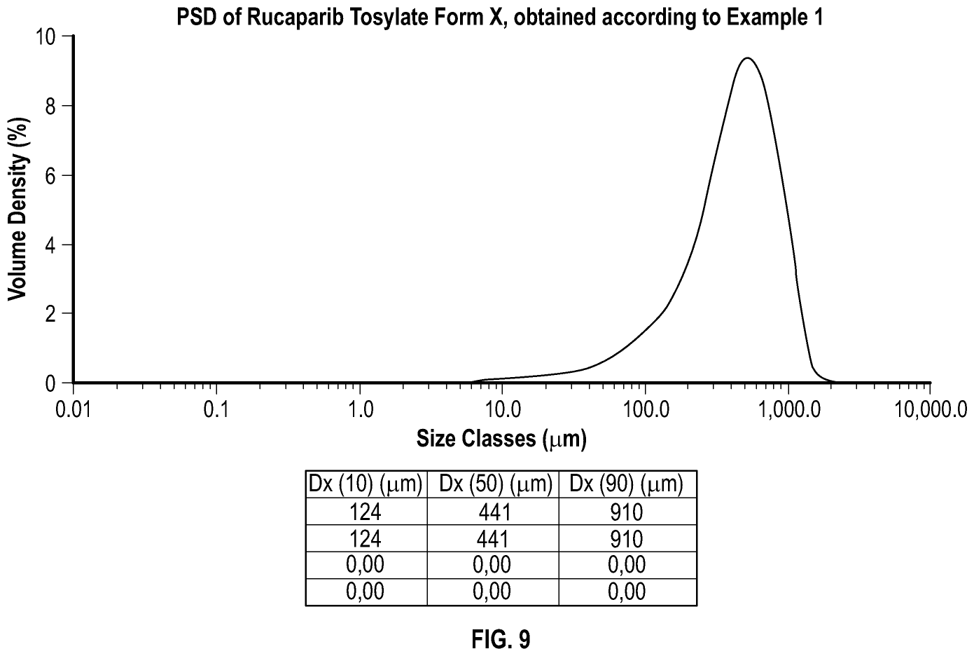

PSD is shown in FIG. 9.

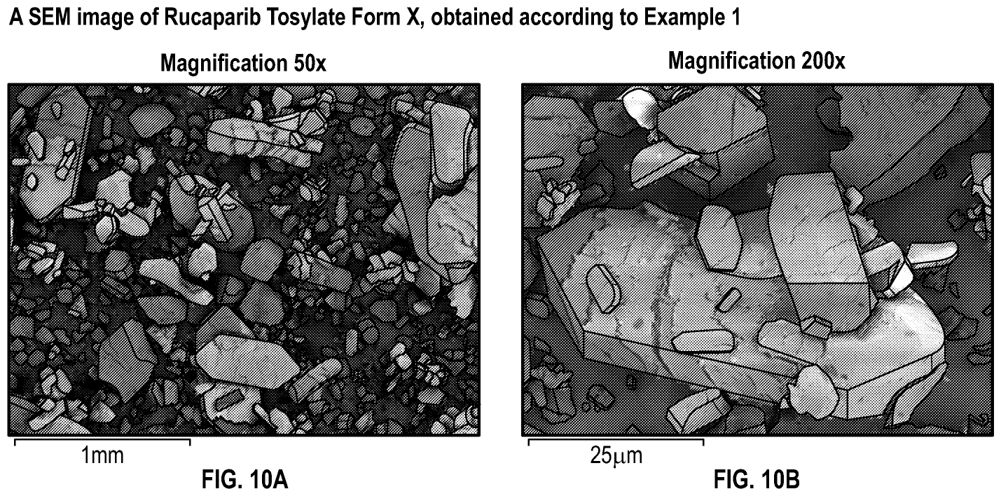

SEM image is shown in FIG. 10.

### Example 2. Preparation of Rucaparib Tosylate Form III Starting from Rucaparib Tosylate Form X

Rucaparib Tosylate Form X (400 mg, prepared according to example 1) was suspended in 2-propanol (4 ml) at temperature of about 50° C. The obtained suspension was stirred for 1 hour at a temperature of about 50° C. and vacuum filtered. The obtained solid was analyzed by XRPD and Rucaparib Tosylate Form III was obtained.

### Example 3. Preparation of Rucaparib Tosylate Form X

Rucaparib base (45 grams, Form C) was suspended in a solvent mixture 2-propanol:water (total volume 570 ml, ratio 50:50 v/v) at room temperature for 25 minutes. p-toluenesulfonic acid monohydrate (27.0 grams, 1.02 eq.) was added to the suspension and the suspension was heated to a temperature of about 80° C. to obtain a solution. The solution was additionally mixed for 10 minutes and then it was cooled to a temperature of about 55° C. over a period of 1 hour, at a controlled cooling rate of about 0.4° C./minute. Rucaparib Tosylate (2.25 grams) was added to the solution and it was mixed for 60 minutes at a temperature of about 55° C. and then it was cooled to a temperature of about 2° C. over a period of about 1.5 hours, at a controlled cooling rate of about 0.6° C./minute. The obtained suspension was mixed for 60 minutes for 2 hours (preferably mixed for 60 minutes at 2° C.) and then the solid was vacuum filtered. A sample was washed with 2×45 ml of 2-propanol and vacuum dried at a temperature of about 80° C. to a constant mass. The obtained solid was analyzed by XRPD and Rucaparib tosylate Form X was obtained. A XRPD pattern is shown in FIG. 4.

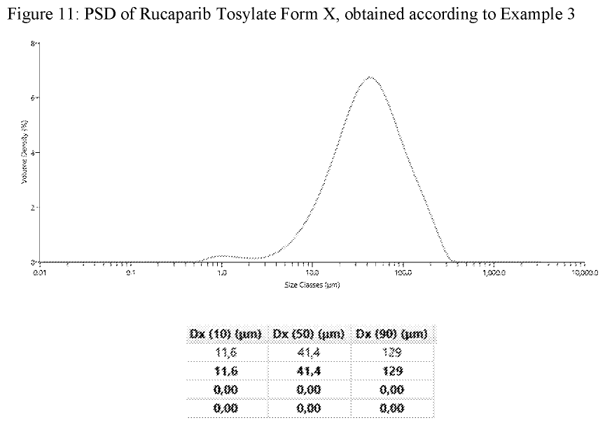

PSD is shown in FIG. 11.

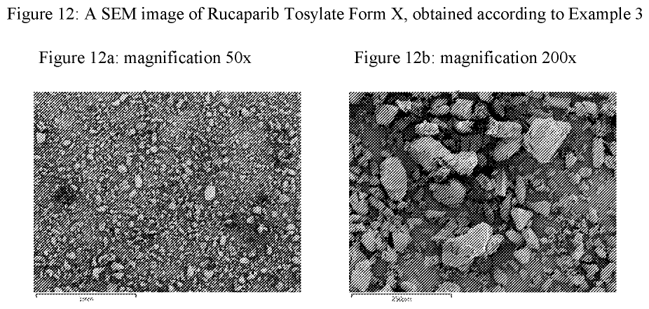

SEM image is shown in FIG. 12.

### Example 4. Preparation of Rucaparib Tosylate Form X

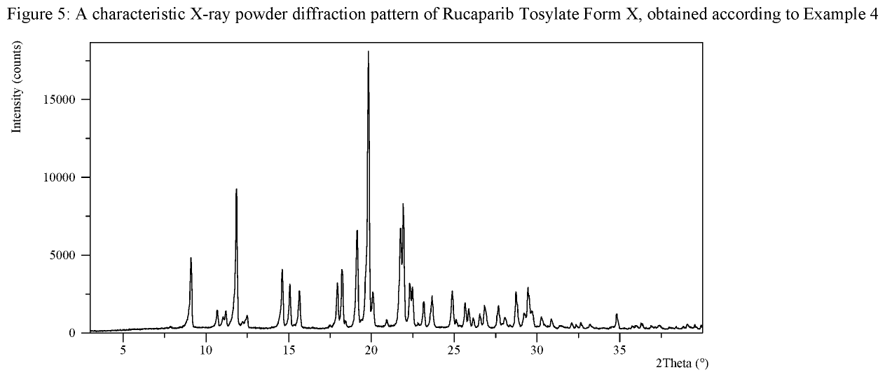

Rucaparib base (3 grams, Form C) and p-toluenesulfonic acid monohydrate (1.76 grams) were suspended in 90 ml of ethanol, 96% (53 w/v) at room temperature. The obtained suspension was mixed for 24 hours at room temperature and the solid was vacuum filtered. The obtained solid was washed with 2×7.5 ml of ethanol, 96% and vacuum dried at a temperature of about 50° C. to a constant mass. A sample was analyzed by XRPD and Rucaparib tosylate Form X was obtained. A XRPD pattern is shown in FIG. 5.

### Example 5. Preparation of Rucaparib Tosylate Form XII

Rucaparib tosylate (4000 mg, Form X) was suspended in 2-propanol (40 ml) at a temperature of about 75° C. for 4 hours. The obtained suspension was allowed to cool to room temperature and additionally mixed for 24 hours. The obtained solid was vacuum filtered and analyzed by XRPD. Rucaparib tosylate Form XII was obtained. A XRPD pattern is shown in FIG. 6.

Rucaparib tosylate Form X used to prepare Form XII in this example, is Form X prepared in example 3.

### Example 6. Preparation of Rucaparib Tosylate Form XIII

Rucaparib tosylate (Form X, 2 mg) was placed in a pin hole aluminum pan. The sample was subjected to thermal treatment in DSC Discovery TA instruments, according to the following steps:
1. Heating of the sample by heating rate 10° C./minute up to temperature of 250° C.,
2. Isothermal heating for 15 minutes at 250° C.,
3. Cooled to room temperature

The obtained solid was analyzed by XRPD. Rucaparib tosylate Form XIII was obtained. A XRPD pattern is shown in FIG. 7.

### Example 7. Preparation of Rucaparib Tosylate Form IX

Rucaparib tosylate (200 mg, Form X) was suspended in toluene (2 ml) at a temperature of about 110° C. for a period of about 10 hours. The obtained suspension was allowed to cool to room temperature and the solid was vacuum filtered. The obtained solid was analyzed by XRPD. Rucaparib tosylate Form IX was obtained. A XRPD pattern is shown in FIG. 8.

### Example 8. Preparation of Rucaparib Tosylate Form X

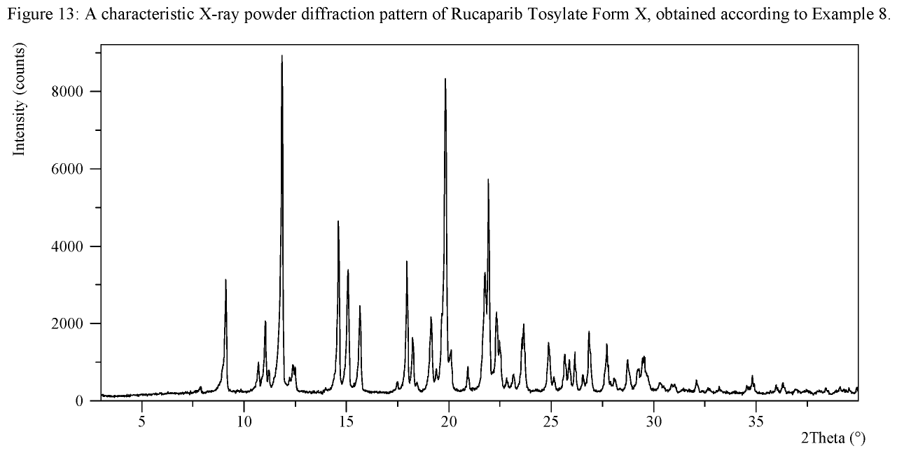

Rucaparib base (50 grams, Form C) was suspended in a solvent mixture of acetonitrile:water (ratio 70:30 v/v, total volume 400 ml) at room temperature for 5 minutes. p-toluenesulfonic acid monohydrate (30 grams, 1.02 eq.) was added to the suspension and it was heated to a temperature of about 80° C. to obtain a solution. The solution was additionally mixed for 10 minutes and then cooled down to a temperature of about 41° C. (rate 0.6° C./min) and a suspension was formed. The suspension was mixed for 60 minutes at temperature of about 41° C. and cooled down to a temperature of about 2° C. over a period of about 90 minutes (cooling rate of 0.4° C./min). After cooling step, the suspension was mixed for about 60 minutes at temperature of about 2° C. and then the solid was vacuum filtered. The sample was washed with 2×50 ml of acetonitrile and vacuum dried at temperature of about 80° C. to a constant mass. The obtained solid was analyzed by XRPD and Rucaparib tosylate Form X was obtained. A XRPD pattern is shown in FIG. 13.

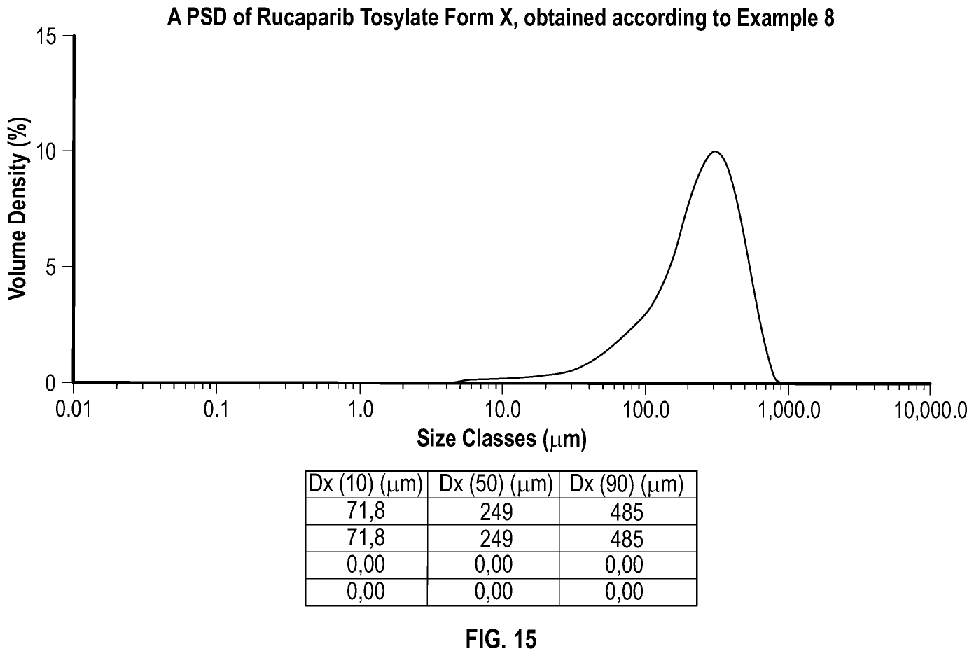

PSD is shown in FIG. 15.

### Example 9. Preparation of Rucaparib Tosylate Form X

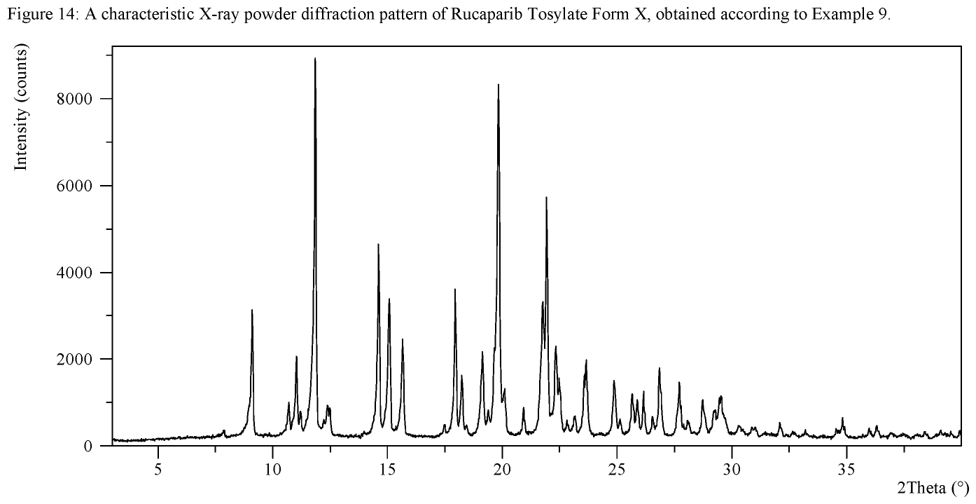

Rucaparib base (131 grams, Form C) was suspended in a solvent mixture of acetone:water (ratio 60:40 v/v, total volume 2400 ml) at room temperature for 5 minutes. p-toluenesulfonic acid monohydrate (78.7 grams, 1.02 eq.) was added to the suspension and it was heated to a temperature of about 55° C. to obtain a solution. The solution was additionally mixed for 10 minutes and was left to cool to a temperature of about 20° C. Crystallization occurred at room temperature and water (450 ml) was added dropwise to the suspension. The suspension was cooled down to a temperature of about 2° C. over a period of about 60 minutes and the suspension was additionally mixed for 120 minutes. The suspension was vacuum filtered and the sample was washed with 2×150 ml of acetone. The solid was vacuum dried at a temperature of about 80° C. for 6 hours. The obtained solid was analyzed by XRPD and Rucaparib tosylate Form X was obtained. A XRPD pattern is shown in FIG. 14.

### Example 10. Preparation of Rucaparib Tosylate Form III

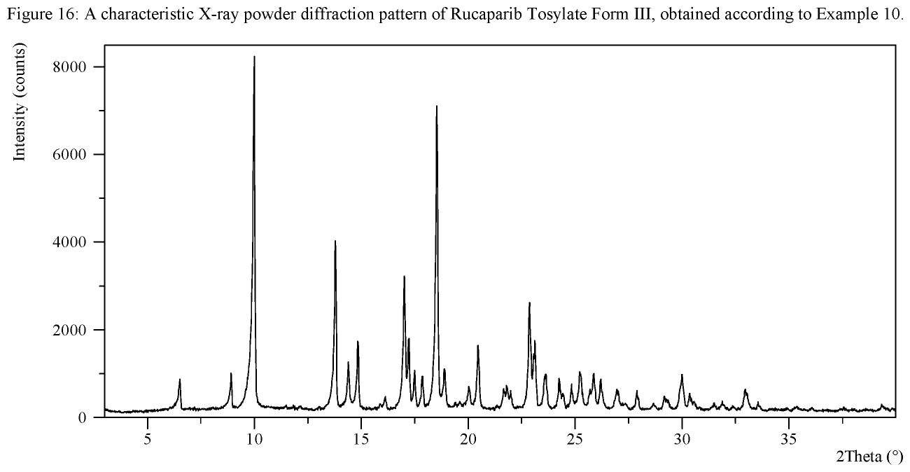

Rucaparib tosylate (25 grams, Form X prepared in a procedure similar to the procedure of example 8) was suspended in acetone (250 ml) at room temperature. The obtained suspension was heated to a temperature of about 50° C. and was mixed for 12 hours. The suspension was cooled down (cooling rate of 0.9° C./min) to room temperature over a period of about 30 minutes and additionally mixed for about 30 minutes. The suspension was vacuum filtered and the sample was washed with acetone (25 ml). The obtained solid was vacuum dried at a temperature of about 80° C. to a constant mass and analyzed by XRPD. Rucaparib tosylate Form III was obtained. A XRPD pattern is shown in FIG. 16.

### Example 11. Preparation of Rucaparib Tosylate Form X

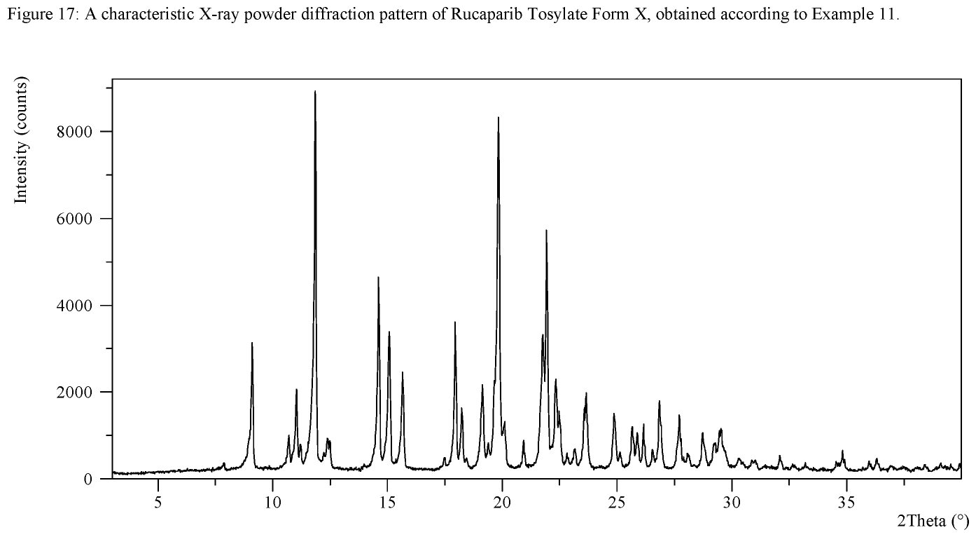

Rucaparib base (50 grams, Form C) was suspended in a solvent mixture of acetonitrile/water (ratio 70:30 v/v, total volume 400 ml) at room temperature for 5 minutes. p-toluenesulfonic acid monohydrate (30.0 grams, 1.02 eq.) was added to the suspension and it was heated to 80° C. to obtain a solution. The solution was additionally mixed for 10 minutes and left to cool to a temperature of about 55° C. Rucaparib tosylate (1.0 gram, Form X) was added to the solution and it was mixed for 60 minutes at a temperature of about 55° C. and then it was actively cooled down in three steps: cooling from 55° C. to 50° C. over 1 hour; cooling from 50° C. to 35° C. over 1 hour and cooling from 35° C. to 2° C. over 1 hour. The obtained suspension was additionally mixed for 60 minutes at temperature of about 0-5° C. and vacuum filtered. The sample was washed with 2×50 ml of acetonitrile and vacuum dried at 80° C. for 5 hours. The obtained solid was analyzed by XRPD and Rucaparib tosylate Form X was obtained. A XRPD pattern is shown in FIG. 17.

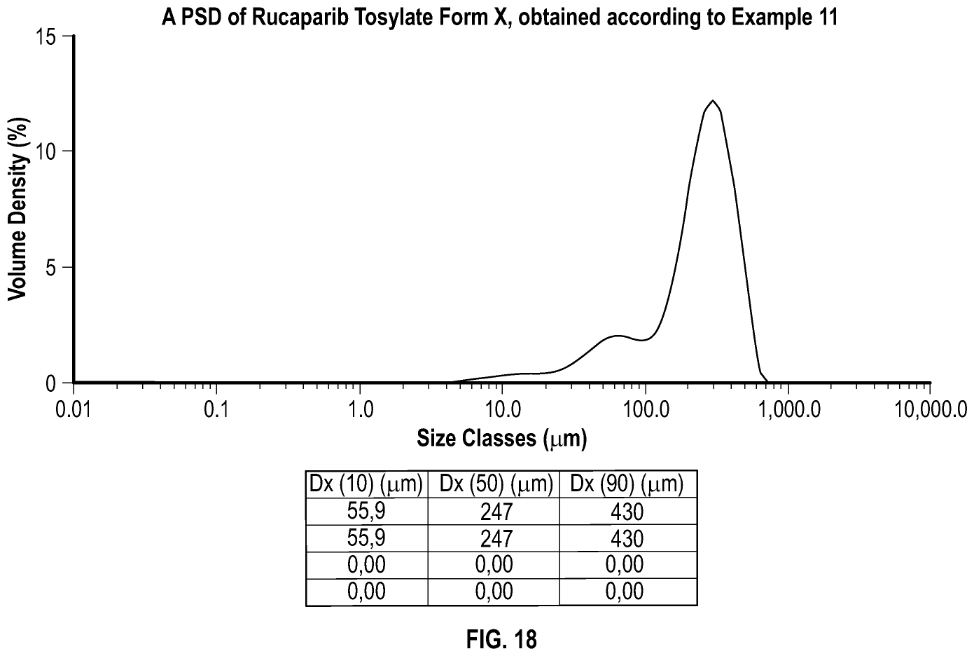

PSD is shown in FIG. 18.

### Example 12. Preparation of Rucaparib Tosylate Form III

Rucaparib tosylate (50 grams, Form X prepared according to example 11) was suspended in acetonitrile (500 ml) and the suspension was heated to a temperature of about 75° C. The suspension was mixed for about 4 hours at a temperature of about 75° C. and cooled down (cooling rate 1.7° C./min) to a temperature of about 22° C. over a period of about 30 minutes. The suspension was additionally mixed for about 30 minutes at room temperature and vacuum filtered. The sample was washed with 50 ml of acetonitrile and vacuum dried at a temperature of about 80° C. to a constant mass. The obtained solid was analyzed by XRPD and Rucaparib tosylate Form III was obtained.

### Example 13. Preparation of Rucaparib Tosylate Form X

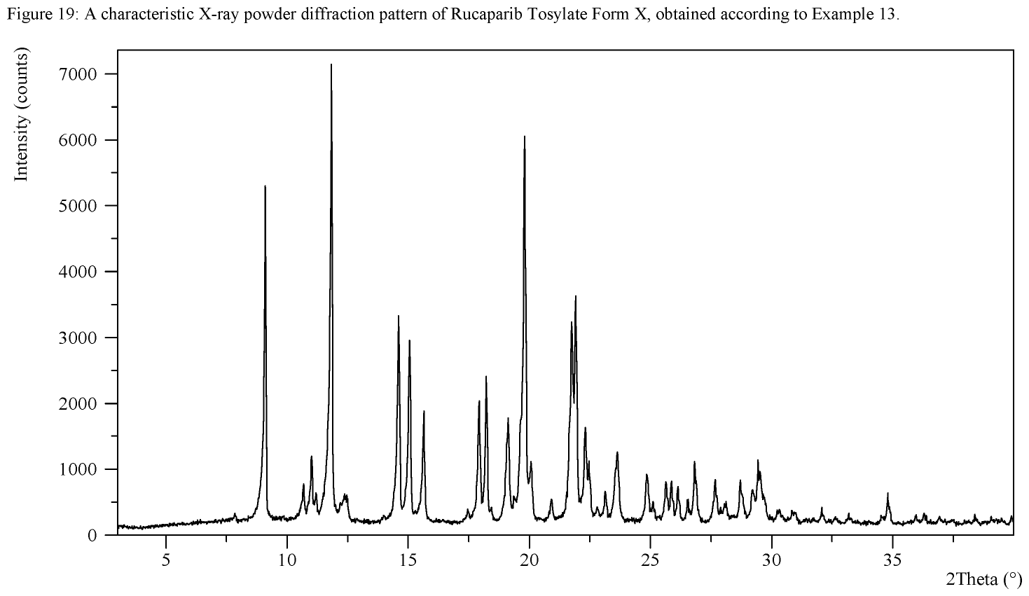

Rucaparib base (26.21 grams, Form C) was suspended in a solvent mixture of acetone/water (ratio 60:40 v/v, total volume 480 ml) at room temperature for 5 minutes. p-toluenesulfonic acid monohydrate (15.73 grams, 1.02 eq.) was added to the suspension and it was heated to a temperature of about 50-55° C. to obtain a solution. The solution was additionally mixed for 10 minutes and cooled down to a temperature of about 2° C. over a period of about 60 minutes. The suspension was additionally mixed for 60 minutes at a temperature of about 2° C. and vacuum filtered. the sample was washed with 2×30 ml of acetone and vacuum dried at a temperature of about 80° C. for 6 hours. The obtained solid was analyzed by XRPD and Rucaparib tosylate Form X was obtained. A XRPD pattern is shown in FIG. 19.

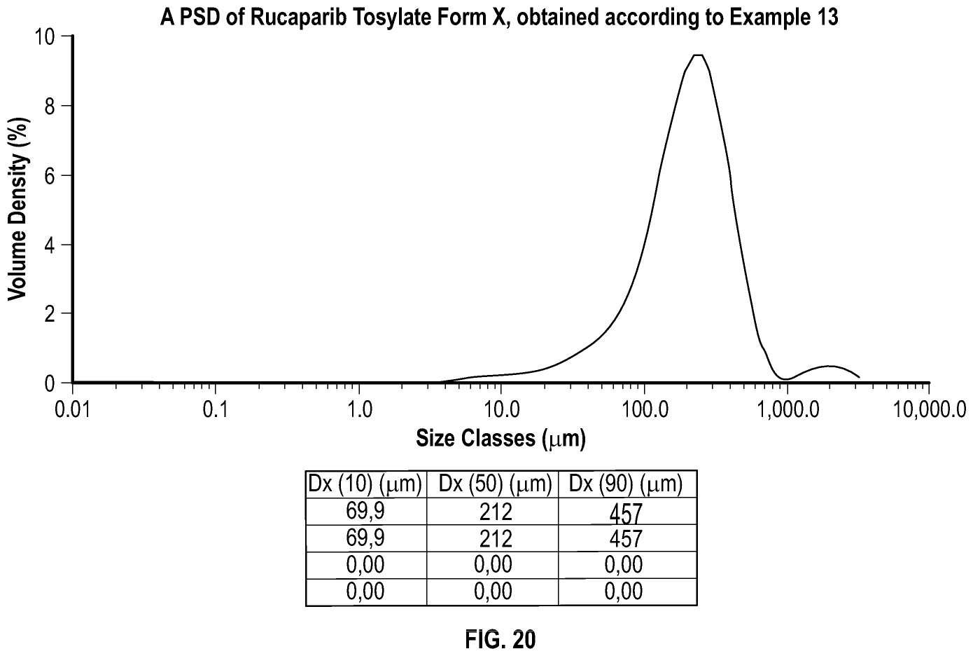

PSD is shown in FIG. 20.

### Example 14. Preparation of Rucaparib Tosylate Form III

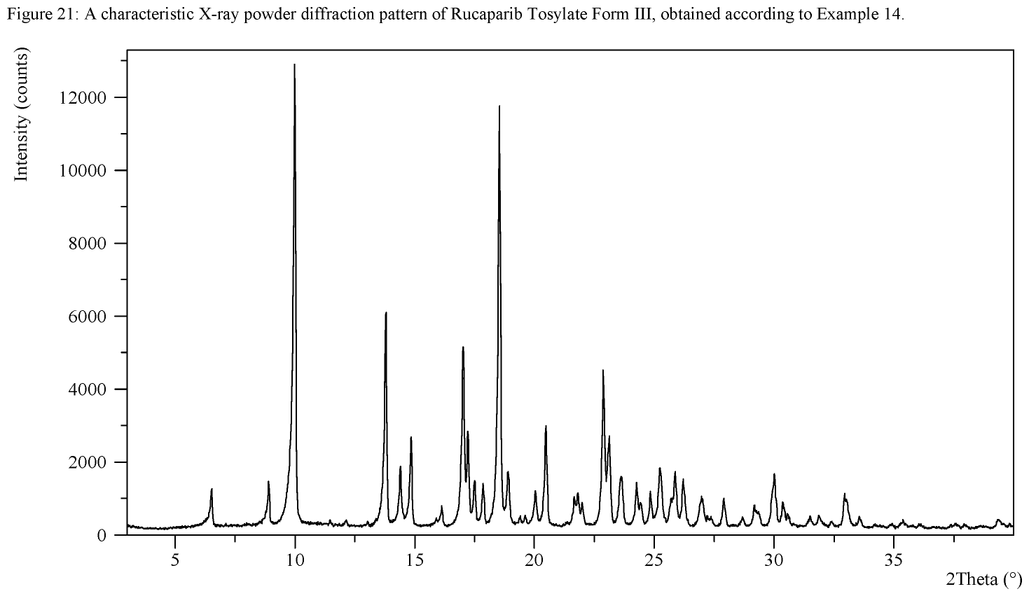

Rucaparib tosylate (30 grams, Form X prepared according to example 14) was suspended in acetone (300 ml) and heated to a temperature of about 50° C. The suspension was mixed for about 12 hours at a temperature of about 50° C. and cooled down to a temperature of about 22° C. over a period of about 30 minutes. The suspension was additionally mixed for about 30 minutes at room temperature and vacuum filtered. The sample was washed with 30 ml of acetone and vacuum dried at a temperature of about 80° C. to a constant mass. The obtained solid was analyzed by XRPD and Rucaparib tosylate Form III was obtained A XRPD pattern is shown in FIG. 21.

### Example 15: Stability Experiments

### Storage Stability at Different Relative Humidities

Samples of Rucaparib tosylate form X (about 200 mg each) were subjected to conditions of different relative humidities at ambient temperature. XRPD analysis was performed on the samples after 7 days. The results are shown in Table 5 below (measured in triplicate):

TABLE 5

| Rucaparib tosylate | Relative humidity | | | | |
|---|---|---|---|---|---|
| form X | 20% | 40% | 60% | 80% | 100% |
| XRPD analysis results | Form X | Form X | Form X | Form X | Form X |

These results demonstrate that Rucaparib tosylate form X is stable after exposure to high and low relative humidity for at least 7 days.

A sample of Rucaparib tosylate form X was subjected to 100% relative humidity at 25° C. XRPD analysis was performed on the sample after 30 days, and confirmed that there was no polymorphic change (measured in triplicate).

### Grinding Experiments

Samples of Rucaparib tosylate form X were subjected to strong grinding, and to solvent drop grinding in water, ethanol (96%) and 2-propanol. Grinding was carried out on the samples alone, or in the presence of ethanol, water and 2-propanol. In these experiments, about 20-30 mg of the sample were placed in a mortar and ground with a pestle for 3 minutes. The solvent, when used, as added to the crystalline material before grinding, in a volume of 10 microlitres. XRPD analysis performed on each of the samples after the grinding experiment, confirmed no change in the starting material (Table 6):

TABLE 6
Rucaparib tosylate form X

| Condition | XRPD analysis results |
|---|---|
| Strong grinding, 3 minutes | Form X |
| Solvent-drop grinding (ethanol) | Form X |
| Solvent-drop grinding (water) | Form X |
| Solvent-drop grinding (2-propanol) | Form X |

The results demonstrate that Rucaparib tosylate form X is resistant to polymorphic changes and is highly suitable for preparing pharmaceutical formulations.

### Exposure to Solvent Vapour

Samples of Rucaparib tosylate form X (20-40 mg) were put in small vials and exposed to solvent vapours (2.5 ml each of ethanol, 96%, water and 2-propanol) at 25° C. XRPD analysis performed on each of the samples after 7 days (Table 7):

TABLE 7
Rucaparib tosylate form X

| Solvent vapour | XRPD analysis results |
|---|---|
| Ethanol, 96% | Form X |
| Water | Form X |
| 2-propanol | Form X |

### Thermal Stability

A sample of Rucaparib tosylate form X was subjected to heating up to 100° C. for 30 minutes. XRPD analysis of the sample confirmed no change in the starting material (Table 8):

TABLE 8
Rucaparib tosylate form X

| Condition | Result |
|---|---|
| Heating 100° C., 30 minutes | Form X |

### Stability to Compression

Samples of Rucaparib tosylate form X were subjected to pressures of 1000 kg and 2000 kg. XRPD analysis was performed on the samples after 1-5 minutes. The results are shown in Table 9 below:

TABLE 9

Pressure

| XRPD analysis results | 1000 kg | 2000 kg |
|---|---|---|
| Rucaparib tosylate form X | No change | No change |

The results demonstrate that Rucaparib tosylate form X is stable under high pressure conditions, making it highly suitable for pharmaceutical processing.

1. Crystalline Rucaparib tosylate form X, which is characterized by data selected from:
a. an X-ray powder diffraction pattern having peaks at 9.1, 11.8, 15.6, 19.8 and 21.9 degrees 2-theta±0.2 degrees 2-theta;
b. a unit cell having the following data:

| | |
|---|---|
| Cell length a | 9.7503(3) Å |
| Cell length b | 15.8393(6) Å |
| Cell length c | 16.1027(5) Å |
| Cell angle alpha | 90° |
| Cell angle beta | 92.509(3)° |
| Cell angle gamma | 90° |
| Cell volume | 2484.48(14) Å3 |
| Z | 4 |
| Symmetry cell setting | monoclinic |
| Symmetry space group name | P21/c |

and
c. a combination of (a) and (b).

2. Crystalline Rucaparib tosylate form X according to claim 1, characterized by an X-ray powder diffraction pattern having peaks at 9.1, 11.8, 15.6, 19.8 and 21.9 degrees 2-theta±0.2 degrees 2-theta, and also having any one, two, three, four or five additional peaks selected from 10.7, 14.6, 17.9, 19.1 and 26.8 degrees 2-theta±0.2 degrees 2-theta.

3. Crystalline Rucaparib tosylate form X according to claim 1, characterized by an X-ray powder diffraction pattern having peaks at 9.1, 10.7, 11.8, 14.6, 15.6, 17.9, 19.1, 19.8, 21.9 and 26.8 degrees 2-theta±0.2 degrees 2-theta; or an X-ray powder diffraction pattern substantially as depicted in FIG. 1.

4. Crystalline Rucaparib tosylate form X according to claim 1, characterized by an X-ray powder diffraction pattern having peaks at 9.1, 10.7, 11.0, 11.2, 11.8, 12.4, 12.5, 14.6, 15.1, 15.6, 17.5, 17.9, 18.2, 19.1, 19.4, 19.6, 19.8, 20.1, 20.9, 21.6, 21.7, 21.9, 22.3, 22.5, 22.8, 23.1, 23.7, 24.9, 25.1, 25.6, 25.9, 26.1, 26.5, 26.8, 27.7, 28.1, 28.7, 29.2, 29.4, 29.5, 29.7, 30.2, 30.8, 31.0, and 34.8 degrees 2-theta±0.2 degrees 2-theta.

5. Crystalline Rucaparib tosylate form X according to claim 1, wherein the crystalline form is isolated.

6. Crystalline Rucaparib tosylate form X according to claim 1, having a water content of about 2.0 wt % to about 5.0 wt %.

7. Crystalline Rucaparib tosylate form X according to claim 1, wherein the crystalline form is a hydrate form having a water content of about 2.0 wt % to about 5.0 wt %.

8. Crystalline Rucaparib tosylate form X according to claim 1, which contains no more than about 20% of any other crystalline forms of Rucaparib tosylate.

9. Crystalline Rucaparib tosylate form X according to claim 1, which contains no more than about 20% of amorphous Rucaparib tosylate.

10. Crystalline Rucaparib tosylate form X according to claim 1, which is chemically pure.

11. A pharmaceutical composition comprising a crystalline Rucaparib tosylate from X according to claim 1 and at least one pharmaceutically acceptable excipient.

12. (canceled)

13. A process comprising combining a crystalline Rucaparib tosylate form X according to claim 1 with at least one pharmaceutically acceptable excipient.

14. A medicament comprising the crystalline Rucaparib tosylate form X according to claim 1.

15. (canceled)

16. A method of treating deleterious BRCA mutation associated advanced ovarian cancer, prostate cancer, fallopian tube cancer or peritoneal cancer, comprising administering a therapeutically effective amount of a crystalline Rucaparib tosylate form X according to claim 1 to a subject in need of treatment.

17. (canceled)

18. (canceled)

19. (canceled)

20. A process for preparing a solid state form of Rucaparib tosylate, Rucaparib tosylate salt and/or solid state form thereof, comprising preparing the crystalline Rucaparib tosylate from X according to claim 1, and converting it to another a solid state form thereof tosylate.

21. A process for preparing crystalline Form III of Rucaparib Tosylate comprising suspending Rucaparib Tosylate Form X according to claim 1, in 2-propanol.

22. A process according to claim 21, wherein the mean PSD of crystalline Form X of Rucaparib Tosylate used to prepare crystalline Form III of Rucaparib Tosylate is of Dx(90) of from about 700 μm to about 960 μm.

23. A process according to claim 21, wherein the mean PSD of crystalline Form X of Rucaparib Tosylate used to prepare crystalline Form III of Rucaparib Tosylate is of Dx(90) from about 800 μm to about 910 μm.

24. A process for preparing crystalline Form III of Rucaparib Tosylate comprising suspending Rucaparib Tosylate Form X according to claim 1 in acetone.

25. A process according to claim 24, wherein the mean PSD of crystalline Form X of Rucaparib Tosylate used to prepare crystalline Form III of Rucaparib Tosylate is of Dx(90) of from 400 μm to about 800 μm.

26. A process according to claim 24, wherein the mean PSD of crystalline Form X of Rucaparib Tosylate used to prepare crystalline Form III of Rucaparib Tosylate is of Dx(90) from about 400 μm to about 600 μm.

27. A process for preparing crystalline Form III of Rucaparib Tosylate comprising suspending Rucaparib Tosylate Form X according to claim 1, in acetonitrile.

28. A process according to claim 27, wherein the mean PSD of crystalline Form X of Rucaparib Tosylate used to prepare crystalline Form III of Rucaparib Tosylate is of Dx(90) of from about 400 μm to about 600 μm.

29. A process according to claim 27, wherein the mean PSD of crystalline Form X of Rucaparib Tosylate used to prepare crystalline Form III of Rucaparib Tosylate is of Dx(90) from about 400 to about 580 μm.

30. A process according to claim 20, further comprising combining the Rucaparib Tosylate form III with at least one pharmaceutically acceptable excipient to prepare a pharmaceutical composition.
